(19) United States
(12) Patent Application Publication
(10) Pub. No.: US 2025/0162156 A1
(43) Pub. Date: May 22, 2025

(54) CONTROL REDIRECTION AND IMAGE PORTING BETWEEN SURGICAL SYSTEMS

(71) Applicant: Cilag GmbH International, Zug (CH)

(72) Inventors: Frederick E. Shelton, IV, Hillsboro, OH (US); Jacqueline Corrigan Aronhalt, Loveland, OH (US); Charles J. Scheib, Loveland, OH (US); Kevin M. Fiebig, Cincinnati, OH (US); Taylor W. Aronhalt, Loveland, OH (US)

(21) Appl. No.: 18/810,082

(22) Filed: Aug. 20, 2024

Related U.S. Application Data
(60) Provisional application No. 63/602,040, filed on Nov. 22, 2023.
(60) Provisional application No. 63/602,028, filed on Nov. 22, 2023.
(60) Provisional application No. 63/601,998, filed on Nov. 22, 2023.
(60) Provisional application No. 63/602,003, filed on Nov. 22, 2023.
(60) Provisional application No. 63/602,006, filed on Nov. 22, 2023.
(60) Provisional application No. 63/602,011, filed on Nov. 22, 2023.
(60) Provisional application No. 63/602,013, filed on Nov. 22, 2023.
(60) Provisional application No. 63/602,037, filed on Nov. 22, 2023.
(60) Provisional application No. 63/602,007, filed on Nov. 22, 2023.

(51) Int. Cl. B25J 9/16; A61B 34/35; A61B 90/00

(52) U.S. Cl. CPC B25J 9/1689; A61B 34/35; A61B 90/37; B25J 9/1697

(57) ABSTRACT

Systems, methods, and instrumentalities associated with shared or surrendered control (e.g., partial or full control) of a surgical system are described. Surgical systems may identify other surgical systems that support image porting and remote control. One of surgical systems may receive from another surgical system a request associated with redirection of imaging and/or a control interface to that surgical system. The surgical system may start sending imaging and/or indication of controls to the other surgical system, which may then display imaging and/or and the control interface of the first surgical system. One of the surgical systems after evaluate control settings may start monitor patient biomarker data based on the evaluated control settings. A surgical system may terminate remote control, for example, based on the patient biomarker data crossing a threshold. The surgical system may send a notification that indicates that it is taking control.

### CROSS-REFERENCE TO RELATED APPLICATIONS

This application claims the benefit of the following, the disclosures of which are incorporated herein by reference in its entirety:
Provisional U.S. Patent Application No. 63/602,040, filed Nov. 22, 2023;
Provisional U.S. Patent Application No. 63/602,028, filed Nov. 22, 2023;
Provisional U.S. Patent Application No. 63/601,998, filed Nov. 22, 2023.
Provisional U.S. Patent Application No. 63/602,003, filed Nov. 22, 2023,
Provisional U.S. Patent Application No. 63/602,006, filed Nov. 22, 2023,
Provisional U.S. Patent Application No. 63/602,011, filed Nov. 22, 2023,
Provisional U.S. Patent Application No. 63/602,013, filed Nov. 22, 2023,
Provisional U.S. Patent Application No. 63/602,037, filed Nov. 22, 2023, and
Provisional U.S. Patent Application No. 63/602,007, filed Nov. 22, 2023.

### BACKGROUND

A surgical procedure may be performed within a surgical environment, such as an operating room. The surgical environment may include many interrelated systems and devices that communicate with each other to perform surgical procedures. Each surgical procedures may use tailored surgical environments with specific systems and/or devices.

Innovative medical technology may include interrelated surgical devices and/or surgical systems that support each other throughout a surgical procedure. The interrelated surgical devices and/or surgical systems may have different capabilities. The interrelated surgical devices and/or surgical systems may be operated remotely (e.g., by a control system) The interrelated surgical devices and/or surgical systems may improve approaches to surgical procedures.

Throughout a surgical procedure, the many interrelated surgical devices/surgical systems may generate and exchange various types and amounts of data between each other to perform various tasks associated with the surgical procedure. Remote operation of surgical systems may pose problems.

### SUMMARY

For example, discovery of smart surgical systems that support image porting and remote control may be performed (e.g., by smart surgical systems in an operating room). A first surgical system may determine that a second surgical system supports image porting and remote control. A first surgical system may receive a request (e.g., second surgical system may send a request) associated with redirection of imaging and a control interface from the first surgical system to the second surgical system (e.g., remote control surgical system). The second surgical system (e.g., based on the request) may receive imaging and indication of controls (e.g., full control or partial control) associated with the first surgical system. The second surgical system may display imaging from the first surgical system and the control interface of the first surgical system (e.g., based on a received set of operational controls (OCs) that the second surgical system is permitted/enabled to change/modify). The second surgical system may request a control setting change associated with the OC of the first surgical system. The first surgical system may determine whether to validate the control setting change.

In examples, the first surgical system may validate the control setting change. The first surgical system may change the control setting (e.g., operating configuration). The first surgical system may send an acknowledgment to the second surgical system indicating the control setting change. The first surgical system may send an additional (e.g., updated) set of OCs that the second surgical system is enabled (e.g., permitted) to change. The second surgical system may display updated imaging from the first surgical system and an updated control interface of the first surgical system based on the received set of OCs that it is permitted to change. In examples, the first surgical system may determine to reject the requested control setting change. The first surgical system may send a NACK and/or a reason for NACK to the second surgical system. The second surgical system may update (e.g., remove) control settings based on the NACK.

In examples, the first surgical system may determine to terminate remote control of the first surgical system by the second surgical system. The first surgical system may evaluate and/or monitor OCs that are set based on the control settings. The first surgical system may monitor data (e.g., patient biomarker data), to determine control settings. The first surgical system may terminate remote control, for example, based on the patient biomarker data. The first surgical system may send a notification that indicates that the first surgical system is taking control. The second surgical system may update (e.g., remove) the control settings and/or imaging based on the received notification.

### BRIEF DESCRIPTIONS OF DRAWINGS

FIG. 1 is a block diagram of a computer-implemented surgical system.

FIG. 2 shows an example surgical system in a surgical operating room.

FIG. 3 illustrates an example surgical hub paired with various systems.

FIG. 4 shows an example situationally aware surgical system.

FIG. 5 illustrates a logical representation of surgical systems and/or surgical imaging systems sharing control, information, and porting imaging using techniques described herein.

FIG. 6 illustrates an example surgical operating room with robotic surgical systems.

FIG. 7 illustrates an example of control redirection where a surgical instrument or a tool is part of a primary surgical system.

FIG. 8 illustrates an example of control redirection where a surgical instrument or a tool is part or a secondary surgical system and separated from a primary surgical system.

FIG. 9 is a flow chart illustrating one robotic surgical system (e.g., a laparoscopic robotic surgical system) performing a surgical procedure in tandem with a second robotic surgical system (e.g., an endoscopic robotic surgical system), and a smart surgical system.

FIG. 10 illustrates an example of a surgical procedure being performed by one robotic surgical system (e.g., a laparoscopic robotic surgical system) working in tandem with a second robotic surgical system (e.g., an endoscopic robotic surgical system), and a smart surgical system.

FIGS. 11A-11B illustrate an exemplary colorectal tumor removal surgical procedure using techniques described herein.

FIG. 12 illustrates an example tumor removal using techniques described herein.

FIG. 13 is a message sequence diagram illustrating control sharing between two surgical systems.

FIG. 14 illustrates an example of a user controlling a first surgical system (e.g., a robotic endoscopic flexible scope) being able to control a second surgical system or a smart surgical system (e.g., an imaging system).

FIG. 15 block diagram illustrating exchangeability of imaging streams and control between various surgical systems and smart surgical systems (e.g., imaging console systems).

### DETAILED DESCRIPTION

A more detailed understanding may be had from the following description, given by way of example in conjunction with the accompanying drawings.

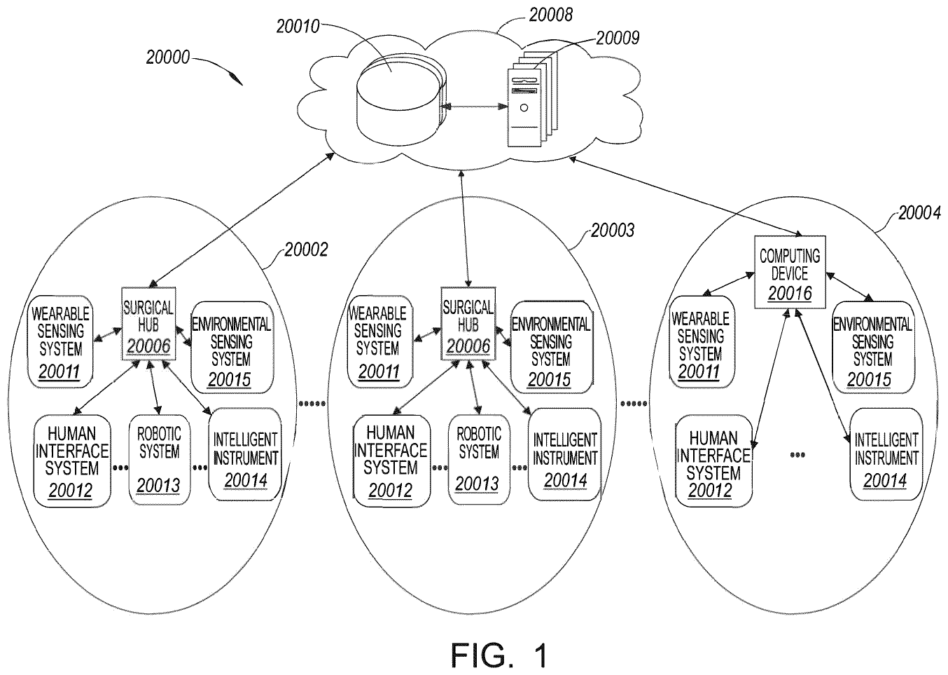

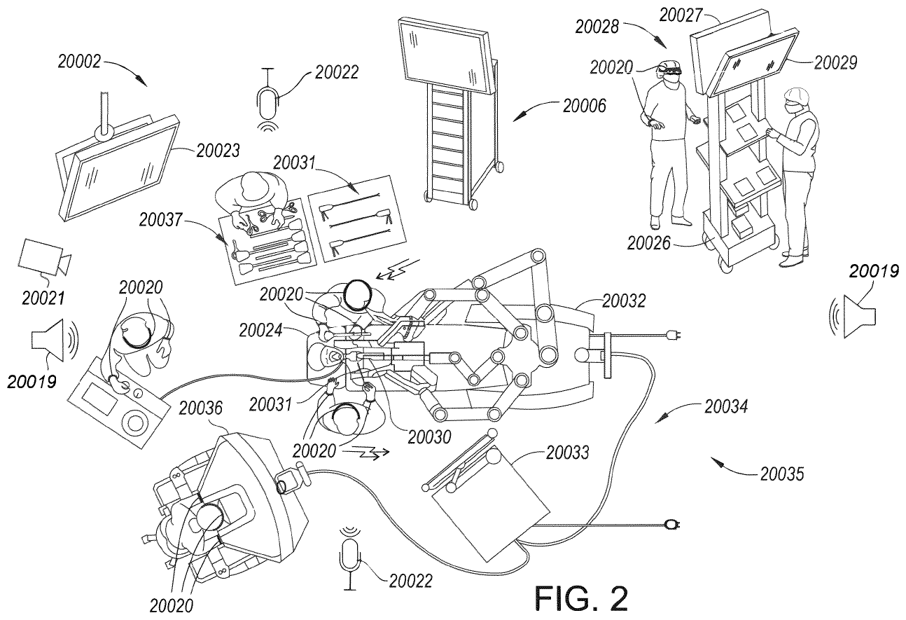

FIG. 1 shows an example computer-implemented surgical system 20000. The example surgical system 20000 may include one or more surgical systems (e.g., surgical sub-systems) 20002, 20003 and 20004. For example, surgical system 20002 may include a computer-implemented interactive surgical system. For example, surgical system 20002 may include a surgical hub 20006 and/or a computing device 20016 in communication with a cloud computing system 20008, for example, as described in FIG. 2. The cloud computing system 20008 may include at least one remote cloud server 20009 and at least one remote cloud storage unit 20010. Example surgical systems 20002, 20003, or 20004 may include one or more wearable sensing systems 20011, one or more environmental sensing systems 20015, one or more robotic systems 20013, one or more intelligent instruments 20014, one or more human interface systems 20012, etc. The human interface system is also referred herein as the human interface device. The wearable sensing system 20011 may include one or more health care professional (HCP) sensing systems, and/or one or more patient sensing systems. The environmental sensing system 20015 may include one or more devices, for example, used for measuring one or more environmental attributes, for example, as further described in FIG. 2. The robotic system 20013 may include a plurality of devices used for performing a surgical procedure, for example, as further described in FIG. 2.

The surgical system 20002 may be in communication with a remote server 20009 that may be part of a cloud computing system 20008. In an example, the surgical system 20002 may be in communication with a remote server 20009 via an internet service provider's cable/FIOS networking node. In an example, a patient sensing system may be in direct communication with a remote server 20009. The surgical system 20002 (and/or various sub-systems, smart surgical instruments, robots, sensing systems, and other computerized devices described herein) may collect data in real-time and transfer the data to cloud computers for data processing and manipulation. It will be appreciated that cloud computing may rely on sharing computing resources rather than having local servers or personal devices to handle software applications.

The surgical system 20002 and/or a component therein may communicate with the remote servers 20009 via a cellular transmission/reception point (TRP) or a base station using one or more of the following cellular protocols: GSM/GPRS/EDGE (2G), UMTS/HSPA (3G), long term evolution (LTE) or 4G, LTE-Advanced (LTE-A), new radio (NR) or 5G, and/or other wired or wireless communication protocols. Various examples of cloud-based analytics that are performed by the cloud computing system 20008, and are suitable for use with the present disclosure, are described in U.S. Patent Application Publication No. US 2019-0206569 A1 (U.S. patent application Ser. No. 16/209,403), titled METHOD OF CLOUD BASED DATA ANALYTICS FOR USE WITH THE HUB, filed Dec. 4, 2018, the disclosure of which is herein incorporated by reference in its entirety.

The surgical hub 20006 may have cooperative interactions with one of more means of displaying the image from the laparoscopic scope and information from one or more other smart devices and one or more sensing systems 20011. The surgical hub 20006 may interact with one or more sensing systems 20011, one or more smart devices, and multiple displays. The surgical hub 20006 may be configured to gather measurement data from the sensing system(s) and send notifications or control messages to the one or more sensing systems 20011. The surgical hub 20006 may send and/or receive information including notification information to and/or from the human interface system 20012. The human interface system 20012 may include one or more human interface devices (HIDs). The surgical hub 20006 may send and/or receive notification information or control information to audio, display and/or control information to various devices that are in communication with the surgical hub.

For example, the sensing systems may include the wearable sensing system 20011 (which may include one or more HCP sensing systems and/or one or more patient sensing systems) and/or the environmental sensing system 20015 shown in FIG. 1. The sensing system(s) may measure data relating to various biomarkers. The sensing system(s) may measure the biomarkers using one or more sensors, for example, photosensors (e.g., photodiodes, photoresistors), mechanical sensors (e.g., motion sensors), acoustic sensors, electrical sensors, electrochemical sensors, thermoelectric sensors, infrared sensors, etc. The sensor(s) may measure the biomarkers as described herein using one of more of the following sensing technologies: photoplethysmography, electrocardiography, electroencephalography, colorimetry, impedimentary, potentiometry, amperometry, etc.

The biomarkers measured by the sensing systems may include, but are not limited to, sleep, core body temperature, maximal oxygen consumption, physical activity, alcohol consumption, respiration rate, oxygen saturation, blood pressure, blood sugar, heart rate variability, blood potential of hydrogen, hydration state, heart rate, skin conductance, peripheral temperature, tissue perfusion pressure, coughing and sneezing, gastrointestinal motility, gastrointestinal tract imaging, respiratory tract bacteria, edema, mental aspects, sweat, circulating tumor cells, autonomic tone, circadian rhythm, and/or menstrual cycle.

The biomarkers may relate to physiologic systems, which may include, but are not limited to, behavior and psychology, cardiovascular system, renal system, skin system, nervous system, gastrointestinal system, respiratory system, endocrine system, immune system, tumor, musculoskeletal system, and/or reproductive system. Information from the biomarkers may be determined and/or used by the computer-implemented patient and the surgical system 20000, for example. The information from the biomarkers may be determined and/or used by the computer-implemented patient and the surgical system 20000 to improve said systems and/or to improve patient outcomes, for example.

The sensing systems may send data to the surgical hub 20006. The sensing systems may use one or more of the following RF protocols for communicating with the surgical hub 20006: Bluetooth, Bluetooth Low-Energy (BLE), Bluetooth Smart, Zigbee, Z-wave, IPv6 Low-power wireless Personal Area Network (6LOWPAN), Wi-FI.

The sensing systems, biomarkers, and physiological systems are described in more detail in U.S. application Ser. No. 17/156,287 (attorney docket number END9290USNP1), titled METHOD OF ADJUSTING A SURGICAL PARAMETER BASED ON BIOMARKER MEASUREMENTS, filed Jan. 22, 2021, the disclosure of which is herein incorporated by reference in its entirety.

The sensing systems described herein may be employed to assess physiological conditions of a surgeon operating on a patient or a patient being prepared for a surgical procedure or a patient recovering after a surgical procedure. The cloud-based computing system 20008 may be used to monitor biomarkers associated with a surgeon or a patient in real-time and to generate surgical plans based at least on measurement data gathered prior to a surgical procedure, provide control signals to the surgical instruments during a surgical procedure, and notify a patient of a complication during post-surgical period.

The cloud-based computing system 20008 may be used to analyze surgical data. Surgical data may be obtained via one or more intelligent instrument(s) 20014, wearable sensing system(s) 20011, environmental sensing system(s) 20015, robotic system(s) 20013 and/or the like in the surgical system 20002. Surgical data may include, tissue states to assess leaks or perfusion of sealed tissue after a tissue sealing and cutting procedure pathology data, including images of samples of body tissue, anatomical structures of the body using a variety of sensors integrated with imaging devices and techniques such as overlaying images captured by multiple imaging devices, image data, and/or the like. The surgical data may be analyzed to improve surgical procedure outcomes by determining if further treatment, such as the application of endoscopic intervention, emerging technologies, a targeted radiation, targeted intervention, and precise robotics to tissue-specific sites and conditions. Such data analysis may employ outcome analytics processing and using standardized approaches may provide beneficial feedback to either confirm surgical treatments and the behavior of the surgeon or suggest modifications to surgical treatments and the behavior of the surgeon.

FIG. 2 shows an example surgical system 20002 in a surgical operating room. As illustrated in FIG. 2, a patient is being operated on by one or more health care professionals (HCPs). The HCPs are being monitored by one or more HCP sensing systems 20020 worn by the HCPs. The HCPs and the environment surrounding the HCPs may also be monitored by one or more environmental sensing systems including, for example, a set of cameras 20021, a set of microphones 20022, and other sensors that may be deployed in the operating room. The HCP sensing systems 20020 and the environmental sensing systems may be in communication with a surgical hub 20006, which in turn may be in communication with one or more cloud servers 20009 of the cloud computing system 20008, as shown in FIG. 1. The environmental sensing systems may be used for measuring one or more environmental attributes, for example, HCP position in the surgical theater, HCP movements, ambient noise in the surgical theater, temperature/humidity in the surgical theater, etc.

As illustrated in FIG. 2, a primary display 20023 and one or more audio output devices (e.g., speakers 20019) are positioned in the sterile field to be visible to an operator at the operating table 20024. In addition, a visualization/notification tower 20026 is positioned outside the sterile field. The visualization/notification tower 20026 may include a first non-sterile human interactive device (HID) 20027 and a second non-sterile HID 20029, which may face away from each other. The HID may be a display or a display with a touchscreen allowing a human to interface directly with the HID. A human interface system, guided by the surgical hub 20006, may be configured to utilize the HIDs 20027, 20029, and 20023 to coordinate information flow to operators inside and outside the sterile field. In an example, the surgical hub 20006 may cause an HID (e.g., the primary HID 20023) to display a notification and/or information about the patient and/or a surgical procedure step. In an example, the surgical hub 20006 may prompt for and/or receive input from personnel in the sterile field or in the non-sterile area. In an example, the surgical hub 20006 may cause an HID to display a snapshot of a surgical site, as recorded by an imaging device 20030, on a non-sterile HID 20027 or 20029, while maintaining a live feed of the surgical site on the primary HID 20023. The snapshot on the non-sterile display 20027 or 20029 can permit a non-sterile operator to perform a diagnostic step relevant to the surgical procedure, for example.

The surgical hub 20006 may be configured to route a diagnostic input or feedback entered by a non-sterile operator at the visualization tower 20026 to the primary display 20023 within the sterile field, where it can be viewed by a sterile operator at the operating table. In an example, the input can be in the form of a modification to the snapshot displayed on the non-sterile display 20027 or 20029, which can be routed to the primary display 20023 by the surgical hub 20006.

Referring to FIG. 2, a surgical instrument 20031 is being used in the surgical procedure as part of the surgical system 20002. The hub 20006 may be configured to coordinate information flow to a display of the surgical instrument(s) 20031. For example, in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. A diagnostic input or feedback entered by a non-sterile operator at the visualization tower 20026 can be routed by the hub 20006 to the surgical instrument display within the sterile field, where it can be viewed by the operator of the surgical instrument 20031. Example surgical instruments that are suitable for use with the surgical system 20002 are described under the heading “Surgical Instrument Hardware” and in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety, for example.

As shown in FIG. 2, the surgical system 20002 can be used to perform a surgical procedure on a patient who is lying down on an operating table 20024 in a surgical operating room 20035. A robotic system 20034 may be used in the surgical procedure as a part of the surgical system 20002. The robotic system 20034 may include a surgeon's console 20036, a patient side cart 20032 (surgical robot), and a surgical robotic hub 20033. The patient side cart 20032 can manipulate at least one removably coupled surgical tool 20037 through a minimally invasive incision in the body of the patient while the surgeon views the surgical site through the surgeon's console 20036. An image of the surgical site can be obtained by a medical imaging device 20030, which can be manipulated by the patient side cart 20032 to orient the imaging device 20030. The robotic hub 20033 can be used to process the images of the surgical site for subsequent display to the surgeon through the surgeon's console 20036.

Other types of robotic systems can be readily adapted for use with the surgical system 20002. Various examples of robotic systems and surgical tools that are suitable for use with the present disclosure are described herein, as well as in U.S. Patent Application Publication No. US 2019-0201137 A1 (U.S. patent application Ser. No. 16/209,407), titled METHOD OF ROBOTIC HUB COMMUNICATION, DETECTION, AND CONTROL, filed Dec. 4, 2018, the disclosure of which is herein incorporated by reference in its entirety.

In various aspects, the imaging device 20030 may include at least one image sensor and one or more optical components. Suitable image sensors may include, but are not limited to, Charge-Coupled Device (CCD) sensors and Complementary Metal-Oxide Semiconductor (CMOS) sensors.

The optical components of the imaging device 20030 may include one or more illumination sources and/or one or more lenses. The one or more illumination sources may be directed to illuminate portions of the surgical field. The one or more image sensors may receive light reflected or refracted from the surgical field, Including light reflected or refracted from tissue and/or surgical instruments.

The illumination source(s) may be configured to radiate electromagnetic energy in the visible spectrum as well as the invisible spectrum. The visible spectrum, sometimes referred to as the optical spectrum or luminous spectrum, is the portion of the electromagnetic spectrum that is visible to (e.g., can be detected by) the human eye and may be referred to as visible light or simply light. A typical human eye will respond to wavelengths in air that range from about 380 nm to about 750 nm.

The invisible spectrum (e.g., the non-luminous spectrum) is the portion of the electromagnetic spectrum that lies below and above the visible spectrum (i.e., wavelengths below about 380 nm and above about 750 nm). The invisible spectrum is not detectable by the human eye. Wavelengths greater than about 750 nm are longer than the red visible spectrum, and they become invisible infrared (IR), microwave, and radio electromagnetic radiation. Wavelengths less than about 380 nm are shorter than the violet spectrum, and they become invisible ultraviolet, x-ray, and gamma ray electromagnetic radiation.

In various aspects, the imaging device 20030 is configured for use in a minimally invasive procedure. Examples of imaging devices suitable for use with the present disclosure include, but are not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope.

The imaging device may employ multi-spectrum monitoring to discriminate topography and underlying structures. A multi-spectral image is one that captures image data within specific wavelength ranges across the electromagnetic spectrum. The wavelengths may be separated by filters or by the use of instruments that are sensitive to particular wavelengths, including light from frequencies beyond the visible light range, e.g., IR and ultraviolet. Spectral imaging can allow extraction of additional information that the human eye fails to capture with its receptors for red, green, and blue. The use of multi-spectral imaging is described in greater detail under the heading “Advanced Imaging Acquisition Module” in U.S. Patent Application Publication No. US 2019-0200844 A1 (U.S. patent application Ser. No. 16/209,385), titled METHOD OF HUB COMMUNICATION, PROCESSING, STORAGE AND DISPLAY, filed Dec. 4, 2018, the disclosure of which is herein incorporated by reference in its entirety. Multi-spectrum monitoring can be a useful tool in relocating a surgical field after a surgical task is completed to perform one or more of the previously described tests on the treated tissue. It is axiomatic that strict sterilization of the operating room and surgical equipment is required during any surgery. The strict hygiene and sterilization conditions required in a “surgical theater,” e.g., an operating or treatment room, necessitate the highest possible sterility of all medical devices and equipment. Part of that sterilization process is the need to sterilize anything that comes in contact with the patient or penetrates the sterile field, including the imaging device 20030 and its attachments and components. It will be appreciated that the sterile field may be considered a specified area, such as within a tray or on a sterile towel, that is considered free of microorganisms, or the sterile field may be considered an area, immediately around a patient, who has been prepared for a surgical procedure. The sterile field may include the scrubbed team members, who are properly attired, and all furniture and fixtures in the area.

Wearable sensing system 20011 illustrated in FIG. 1 may include one or more HCP sensing systems 20020 as shown in FIG. 2. The HCP sensing systems 20020 may include sensing systems to monitor and detect a set of physical states and/or a set of physiological states of a healthcare personnel (HCP). An HCP may be a surgeon or one or more healthcare personnel assisting the surgeon or other healthcare service providers in general. In an example, an HCP sensing system 20020 may measure a set of biomarkers to monitor the heart rate of an HCP. In an example, an HCP sensing system 20020 worn on a surgeon's wrist (e.g., a watch or a wristband) may use an accelerometer to detect hand motion and/or shakes and determine the magnitude and frequency of tremors. The sensing system 20020 may send the measurement data associated with the set of biomarkers and the data associated with a physical state of the surgeon to the surgical hub 20006 for further processing.

The environmental sensing system(s) 20015 shown in FIG. 1 may send environmental information to the surgical hub 20006. For example, the environmental sensing system(s) 20015 may include a camera 20021 for detecting hand/body position of an HCP. The environmental sensing system(s) 20015 may include microphones 20022 for measuring the ambient noise in the surgical theater. Other environmental sensing system(s) 20015 may include devices, for example, a thermometer to measure temperature and a hygrometer to measure humidity of the surroundings in the surgical theater, etc. The surgeon biomarkers may include one or more of the following: stress, heart rate, etc. The environmental measurements from the surgical theater may include ambient noise level associated with the surgeon or the patient, surgeon and/or staff movements, surgeon and/or staff attention level, etc. The surgical hub 20006, alone or in communication with the cloud computing system, may use the surgeon biomarker measurement data and/or environmental sensing information to modify the control algorithms of hand-held instruments or the averaging delay of a robotic interface, for example, to minimize tremors.

The surgical hub 20006 may use the surgeon biomarker measurement data associated with an HCP to adaptively control one or more surgical instruments 20031. For example, the surgical hub 20006 may send a control program to a surgical instrument 20031 to control its actuators to limit or compensate for fatigue and use of fine motor skills. The surgical hub 20006 may send the control program based on situational awareness and/or the context on importance or criticality of a task. The control program may instruct the instrument to alter operation to provide more control when control is needed.

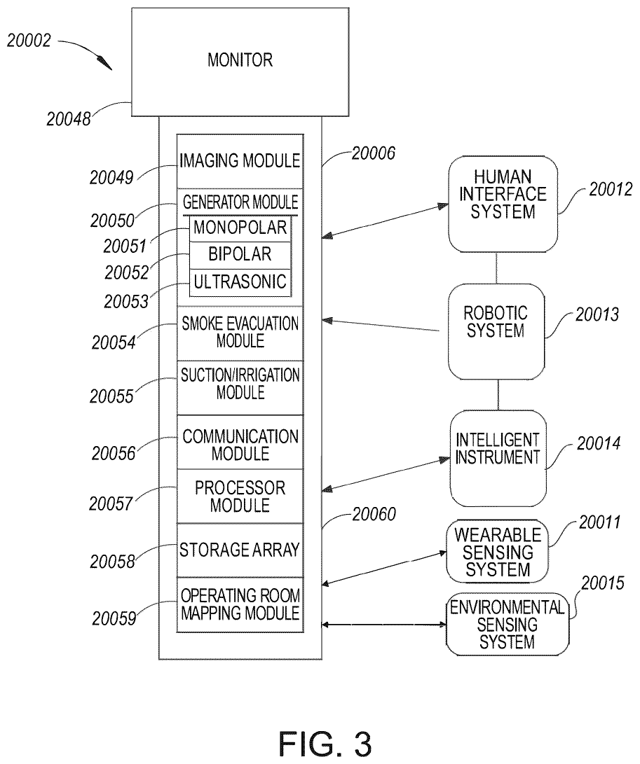

FIG. 3 shows an example surgical system 20002 with a surgical hub 20006. The surgical hub 20006 may be paired with, via a modular control, a wearable sensing system 20011, an environmental sensing system 20015, a human interface system 20012, a robotic system 20013, and an intelligent instrument 20014. The hub 20006 includes a display 20048, an imaging module 20049, a generator module 20050 (e.g., an energy generator), a communication module 20056, a processor module 20057, a storage array 20058, and an operating-room mapping module 20059. In certain aspects, as illustrated in FIG. 3, the hub 20006 further includes a smoke evacuation module 20054 and/or a suction/irrigation module 20055. The various modules and systems may be connected to the modular control either directly via a router or via the communication module 20056. The operating theater devices may be coupled to cloud computing resources and data storage via the modular control. The human interface system 20012 may include a display sub-system and a notification sub-system.

The modular control may be coupled to non-contact sensor module. The non-contact sensor module may measure the dimensions of the operating theater and generate a map of the surgical theater using, ultrasonic, laser-type, and/or the like, non-contact measurement devices. Other distance sensors can be employed to determine the bounds of an operating room. An ultrasound-based non-contact sensor module may scan the operating theater by transmitting a burst of ultrasound and receiving the echo when it bounces of the perimeter walls of an operating theater as described under the heading “Surgical Hub Spatial Awareness Within an Operating Room” in U.S. Provisional Patent Application Ser. No. 62/611,341, titled INTERACTIVE SURGICAL PLATFORM, filed Dec. 28, 2017, which is herein incorporated by reference in its entirety. The sensor module may be configured to determine the size of the operating theater and to adjust Bluetooth-pairing distance limits. A laser-based non-contact sensor module may scan the operating theater by transmitting laser light pulses, receiving laser light pulses that bounce off the perimeter walls of the operating theater, and comparing the phase of the transmitted pulse to the received pulse to determine the size of the operating theater and to adjust Bluetooth pairing distance limits, for example.

During a surgical procedure, energy application to tissue, for sealing and/or cutting, may be associated with smoke evacuation, suction of excess fluid, and/or irrigation of the tissue. Fluid, power, and/or data lines from different sources may be entangled during the surgical procedure. Valuable time can be lost addressing this issue during a surgical procedure. Detangling the lines may necessitate disconnecting the lines from their respective modules, which may require resetting the modules. The hub modular enclosure 20060 may offer a unified environment for managing the power, data, and fluid lines, which reduces the frequency of entanglement between such lines.

Energy may be applied to tissue at a surgical site. The surgical hub 20006 may include a hub enclosure 20060 and a combo generator module slidably receivable in a docking station of the hub enclosure 20060. The docking station may include data and power contacts. The combo generator module may include two or more of: an ultrasonic energy generator component, a bipolar RF energy generator component, or a monopolar RF energy generator component that are housed in a single unit. The combo generator module may include a smoke evacuation component, at least one energy delivery cable for connecting the combo generator module to a surgical instrument, at least one smoke evacuation component configured to evacuate smoke, fluid, and/or particulates generated by the application of therapeutic energy to the tissue, and a fluid line extending from the remote surgical site to the smoke evacuation component. The fluid line may be a first fluid line, and a second fluid line may extend from the remote surgical site to a suction and irrigation module 20055 slidably received in the hub enclosure 20060. The hub enclosure 20060 may include a fluid interface.

The combo generator module may generate multiple energy types for application to the tissue. One energy type may be more beneficial for cutting the tissue, while another different energy type may be more beneficial for sealing the tissue. For example, a bipolar generator can be used to seal the tissue while an ultrasonic generator can be used to cut the sealed tissue. Aspects of the present disclosure present a solution where a hub modular enclosure 20060 is configured to accommodate different generators and facilitate an interactive communication therebetween. The hub modular enclosure 20060 may enable the quick removal and/or replacement of various modules.

The modular surgical enclosure may include a first energy-generator module, configured to generate a first energy for application to the tissue, and a first docking station comprising a first docking port that includes first data and power contacts, wherein the first energy-generator module is slidably movable into an electrical engagement with the power and data contacts and wherein the first energy-generator module is slidably movable out of the electrical engagement with the first power and data contacts. The modular surgical enclosure may include a second energy-generator module configured to generate a second energy, different than the first energy, for application to the tissue, and a second docking station comprising a second docking port that includes second data and power contacts, wherein the second energy generator module is slidably movable into an electrical engagement with the power and data contacts, and wherein the second energy-generator module is slidably movable out of the electrical engagement with the second power and data contacts. In addition, the modular surgical enclosure also includes a communication bus between the first docking port and the second docking port, configured to facilitate communication between the first energy-generator module and the second energy-generator module.

Referring to FIG. 3, the hub modular enclosure 20060 may allow the modular integration of a generator module 20050, a smoke evacuation module 20054, and a suction/irrigation module 20055. The hub modular enclosure 20060 may facilitate interactive communication between the modules 20059, 20054, and 20055. The generator module 20050 can be with integrated monopolar, bipolar, and ultrasonic components supported in a single housing unit slidably insertable into the hub modular enclosure 20060. The generator module 20050 may connect to a monopolar device 20051, a bipolar device 20052, and an ultrasonic device 20053. The generator module 20050 may include a series of monopolar, bipolar, and/or ultrasonic generator modules that interact through the hub modular enclosure 20060. The hub modular enclosure 20060 may facilitate the insertion of multiple generators and interactive communication between the generators docked into the hub modular enclosure 20060 so that the generators would act as a single generator.

A surgical data network having a set of communication hubs may connect the sensing system(s), the modular devices located in one or more operating theaters of a healthcare facility, a patient recovery room, or a room in a healthcare facility specially equipped for surgical operations, to the cloud computing system 20008.

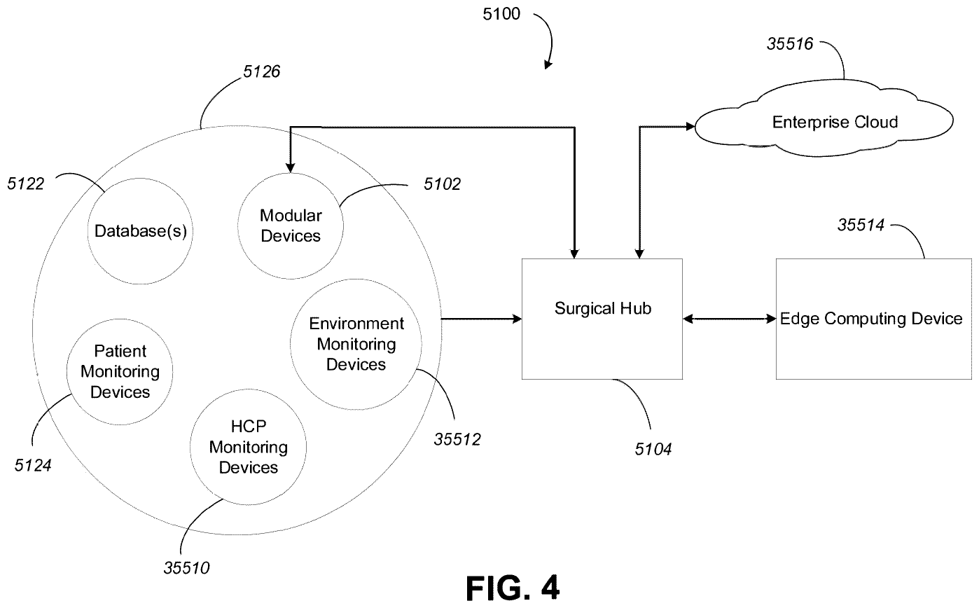

FIG. 4 illustrates a diagram of a situationally aware surgical system 5100. The data sources 5126 may include, for example, the modular devices 5102, databases 5122 (e.g., an EMR database containing patient records), patient monitoring devices 5124 (e.g., a blood pressure (BP) monitor and an electrocardiography (EKG) monitor), HCP monitoring devices 35510, and/or environment monitoring devices 35512. The modular devices 5102 may include sensors configured to detect parameters associated with the patient, HCPs and environment and/or the modular device itself. The modular devices 5102 may include one or more intelligent instrument(s) 20014. The surgical hub 5104 may derive the contextual information pertaining to the surgical procedure from the data based upon, for example, the particular combination(s) of received data or the particular order in which the data is received from the data sources 5126. The contextual information inferred from the received data can include, for example, the type of surgical procedure being performed, the particular step of the surgical procedure that the surgeon is performing, the type of tissue being operated on, or the body cavity that is the subject of the procedure. This ability by some aspects of the surgical hub 5104 to derive or infer information related to the surgical procedure from received data can be referred to as “situational awareness.” For example, the surgical hub 5104 can incorporate a situational awareness system, which may be the hardware and/or programming associated with the surgical hub 5104 that derives contextual information pertaining to the surgical procedure from the received data and/or a surgical plan information received from the edge computing system 35514 or an enterprise cloud server 35516. The contextual information derived from the data sources 5126 may include, for example, what step of the surgical procedure is being performed, whether and how a particular modular device 5102 is being used, and the patient's condition.

The surgical hub 5104 may be connected to various databases 5122 to retrieve therefrom data regarding the surgical procedure that is being performed or is to be performed. In one exemplification of the surgical system 5100, the databases 5122 may include an EMR database of a hospital. The data that may be received by the situational awareness system of the surgical hub 5104 from the databases 5122 may include, for example, start (or setup) time or operational information regarding the procedure (e.g., a segmentectomy in the upper right portion of the thoracic cavity). The surgical hub 5104 may derive contextual information regarding the surgical procedure from this data alone or from the combination of this data and data from other data sources 5126.

The surgical hub 5104 may be connected to (e.g., paired with) a variety of patient monitoring devices 5124. In an example of the surgical system 5100, the patient monitoring devices 5124 that can be paired with the surgical hub 5104 may include a pulse oximeter (SpO2 monitor) 5114, a BP monitor 5116, and an EKG monitor 5120. The perioperative data that is received by the situational awareness system of the surgical hub 5104 from the patient monitoring devices 5124 may include, for example, the patient's oxygen saturation, blood pressure, heart rate, and other physiological parameters. The contextual information that may be derived by the surgical hub 5104 from the perioperative data transmitted by the patient moni-toring devices 5124 may include, for example, whether the patient is located in the operating theater or under anesthesia. The surgical hub 5104 may derive these inferences from data from the patient monitoring devices 5124 alone or in combination with data from other data sources 5126 (e.g., the ventilator 5118).

The surgical hub 5104 may be connected to (e.g., paired with) a variety of modular devices 5102. In one exemplification of the surgical system 5100, the modular devices 5102 that are paired with the surgical hub 5104 may Include a smoke evacuator, a medical imaging device such as the imaging device 20030 shown in FIG. 2, an insufflator, a combined energy generator (for powering an ultrasonic surgical instrument and/or an RF electrosurgical instrument), and a ventilator.

The perioperative data received by the surgical hub 5104 from the medical imaging device may include, for example, whether the medical imaging device is activated and a video or image feed. The contextual information that is derived by the surgical hub 5104 from the perioperative data sent by the medical imaging device may include, for example, whether the procedure is a VATS procedure (based on whether the medical imaging device is activated or paired to the surgical hub 5104 at the beginning or during the course of the procedure). The image or video data from the medical imaging device (or the data stream representing the video for a digital medical imaging device) may be processed by a pattern recognition system or a machine learning system to recognize features (e.g., organs or tissue types) in the field of view (FOY) of the medical imaging device, for example. The contextual information that is derived by the surgical hub 5104 from the recognized features may include, for example, what type of surgical procedure (or step thereof) is being performed, what organ is being operated on, or what body cavity is being operated in.

The situational awareness system of the surgical hub 5104 may derive the contextual information from the data received from the data sources 5126 in a variety of different ways. For example, the situational awareness system can include a pattern recognition system, or machine learning system (e.g., an artificial neural network), that has been trained on training data to correlate various inputs (e.g., data from database(s) 5122, patient monitoring devices 5124, modular devices 5102, HCP monitoring devices 35510, and/or environment monitoring devices 35512) to corresponding contextual information regarding a surgical procedure. For example, a machine learning system may accurately derive contextual information regarding a surgical procedure from the provided inputs. In examples, the situational awareness system can include a lookup table storing pre-characterized contextual information regarding a surgical procedure in association with one or more inputs (or ranges of inputs) corresponding to the contextual information. In response to a query with one or more inputs, the lookup table can return the corresponding contextual information for the situational awareness system for controlling the modular devices 5102. In examples, the contextual information received by the situational awareness system of the surgical hub 5104 can be associated with a particular control adjustment or set of control adjustments for one or more modular devices 5102. In examples, the situational awareness system can include a machine learning system, lookup table, or other such system, which may generate or retrieve one or more control adjustments for one or more modular devices 5102 when provided the contextual information as input.

For example, based on the data sources 5126, the situationally aware surgical hub 5104 may determine what type of tissue was being operated on. The situationally aware surgical hub 5104 can infer whether a surgical procedure being performed is a thoracic or an abdominal procedure, allowing the surgical hub 5104 to determine whether the tissue clamped by an end effector of the surgical stapling and cutting instrument is lung (for a thoracic procedure) or stomach (for an abdominal procedure) tissue. The situationally aware surgical hub 5104 may determine whether the surgical site is under pressure (by determining that the surgical procedure is utilizing insufflation) and determine the procedure type, for a consistent amount of smoke evacuation for both thoracic and abdominal procedures. Based on the data sources 5126, the situationally aware surgical hub 5104 could determine what step of the surgical procedure is being performed or will subsequently be performed.

The situationally aware surgical hub 5104 could determine what type of surgical procedure is being performed and customize the energy level according to the expected tissue profile for the surgical procedure. The situationally aware surgical hub 5104 may adjust the energy level for the ultrasonic surgical instrument or RF electrosurgical instrument throughout the course of a surgical procedure, rather than just on a procedure-by-procedure basis.

In examples, data can be drawn from additional data sources 5126 to improve the conclusions that the surgical hub 5104 draws from one data source 5126. The situationally aware surgical hub 5104 could augment data that it receives from the modular devices 5102 with contextual information that it has built up regarding the surgical procedure from other data sources 5126.

The situational awareness system of the surgical hub 5104 can consider the physiological measurement data to provide additional context in analyzing the visualization data. The additional context can be useful when the visualization data may be inconclusive or incomplete on its own.

The situationally aware surgical hub 5104 could determine whether the surgeon (or other HCP(s) was making an error or otherwise deviating from the expected course of action during the course of a surgical procedure. For example, the surgical hub 5104 may determine the type of surgical procedure being performed, retrieve the corresponding list of steps or order of equipment usage (e.g., from a memory), and compare the steps being performed or the equipment being used during the course of the surgical procedure to the expected steps or equipment for the type of surgical procedure that the surgical hub 5104 determined is being performed. The surgical hub 5104 can provide an alert indicating that an unexpected action is being performed or an unexpected device is being utilized at the particular step in the surgical procedure.

The surgical instruments (and other modular devices 5102) may be adjusted for the particular context of each surgical procedure (such as adjusting to different tissue types) and validating actions during a surgical procedure. Next steps, data, and display adjustments may be provided to surgical instruments (and other modular devices 5102) in the surgical theater according to the specific context of the procedure.

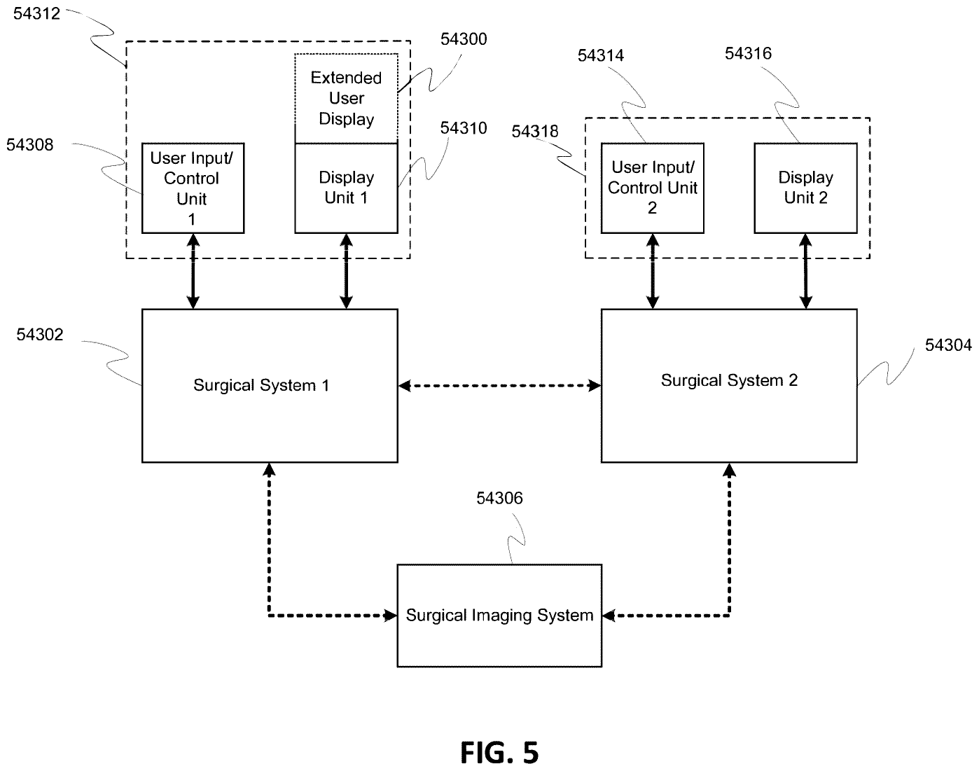

FIG. 5 illustrates a logical representation of surgical systems and/or surgical imaging systems sharing control, information, and porting imaging, as described herein. Surgical systems may include laparoscopic robotic surgical systems, endoscopic robotic surgical systems, etc. that may be used for carrying out minimally invasive surgical procedures. The laparoscopic robotic surgical systems may include a smart robotic arm coupled surgical device, a multi-arm robot, The endoscopic robotic surgical systems may include a smart flexible endoscope, etc. The surgical imaging systems may include a magnetic resonance imaging (MRI) system, a computed tomography (CT) imaging system (e.g., a cone-beam CT scanner), a robotic bronchoscope.

As illustrated in FIG. 5, each of the surgical systems (surgical system 1 54302 and surgical system 2 54304) may be connected with respective user input/control units (54308 and 54314) and display units (54310 and 54316). In an example, the user input/control unit 54308 and display unit 54310 may be separate physical units. In an example, the user input/control unit 54308 and display unit 54310 may be a combined physical unit 54312, and the user input/control unit 54314 and display unit 54316 may be a combined physical unit 54318.

In an example, the functional user controls of surgical system 2 54304 may be displayed and interacted with on surgical system 1 54302. This function may enable an HCP controlling surgical system 1 54302 to request movements, activations, or operation of surgical system 2 without surgical system 2 surrendering internal operational control of the equipment to surgical system 1.

In FIG. 5, the sub-system or internal control of each of the surgical system 1 54302 and the surgical system 2 54304 may be operating in a default mode (e.g., in normal state). The surgical system 1 54302 may operate and control its system fully independent of any other surgical systems or other smart systems that may be present in its vicinity (e.g., in an operating room). The surgical system 1 may be controlled by a healthcare professional (HCP) by interacting with the user input/control unit 1 54308. Similarly, the surgical system 2 may also operate and control its system fully independent of all other surgical systems or other smart systems that may be present in its vicinity.

In an example, the surgical system 1 54302 may be allowed to request control of the surgical system 2 54304. For example, an HCP controlling the surgical system 1 54302 may be allowed as a user proxy to operate the surgical system 2. In such cases, the HCP operating the surgical system 1 54302 may establish a user proxy with the surgical system 2 54304. Once the HCP is established as a user proxy, the surgical system 1 54302 may generate command requests associated with the surgical system 2 54304. The command requests generated by the surgical system 1 54302 may be sent to the surgical system 2 54304. The command requests may include control information that may be used for controlling one or more aspects or functions associated with surgical system 2 54304. A command request generated and sent from surgical system 1 to surgical system 2 may include control information for controlling movements, activations, and/or operations associated with surgical system 2 54304. For example, a command request generated by surgical system 1 54302 may be sent to the surgical system 2 54304 for controlling user interface controller of surgical system 2 54304 (e.g., a device for entering data, a device for moving pointer on the user interface of surgical system 2 54304, a controller interface for controlling cameras, light sources, and other sensors associated with surgical system 2 54304, etc.)

After establishing the user proxy, the display unit 1 54310 associated with the surgical system 1 54302 may be modified in order to accommodate user display or a part of the user display from the surgical system 2 54304. In an example, an extended user display 54300 may be added to the display unit 1 54310 for displaying the information or a part of the information being displayed at display unit 2 54316 of the surgical system 2 54304. The information or a part of the information being displayed by display unit 2 may be exported and/or streamed from surgical system 2 54304 and displayed on the display unit 1 54310. In an example, imaging data may be ported from surgical system 2 and displayed on the display unit 1 54310 or the extended user display 54300 of surgical system 1 54302.

In another example, imaging data may be ported (e.g., streamed) from the surgical imaging system 54306 and processed and displayed on the display unit 1 54310 or the extended user display 54300 of surgical system 1 54302 and/or display unit 2 54316 of surgical system 2 54304.

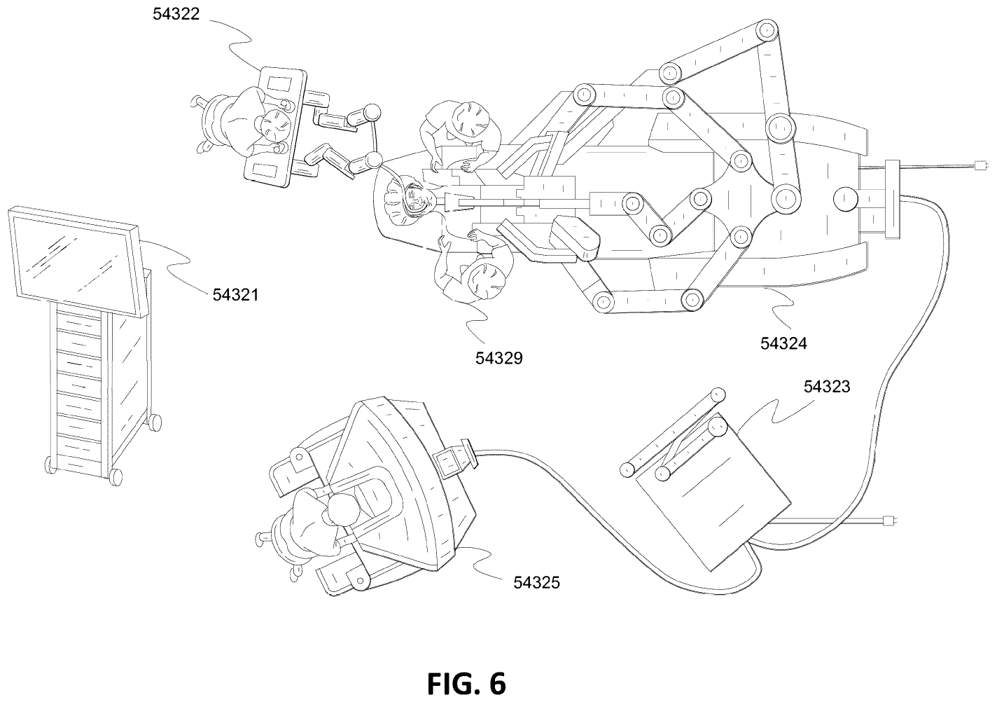

FIG. 6 illustrates an example surgical operating room with robotic surgical systems or instruments and other surgical instruments that may be part of the surgical hub 54321. As illustrated in FIG. 6, a surgical procedure (e.g., a thoracic lung segmentectomy) may be performed on a patient using an endoscopic robotic surgical system 54322 controlled by one HCP and a laparoscopic robotic surgical systems (e.g., a smart robotic arm 54323 and/or a multi-arm robot 54324) that maybe controlled by another HCP using a controller 54325. In an exemplary setup, one or more of the systems shown in FIG. 6 may be configured for performing a surgical procedure. In such an setup, an HCP (e.g., the lead HCP) may control the laparoscopic robotic system 54324 and/or the single arm robotic surgical system 54323 using the console 54325. The endoscopic robotic system 54322 may be controlled by a second HCP (e.g., an assisting HCP), as illustrated in FIG. 6.

In examples, the lead HCP controlling the one or two laparoscopic robotic surgical systems may also send commands to the endoscopic robotic surgical system to controls some aspects of the endoscopic robotic surgical system. The lead HCP may control the endoscopic robotic surgical system either locally or remotely with or without the help of the second HCP 54329. In examples, the second HCP may be in vicinity of the patient.

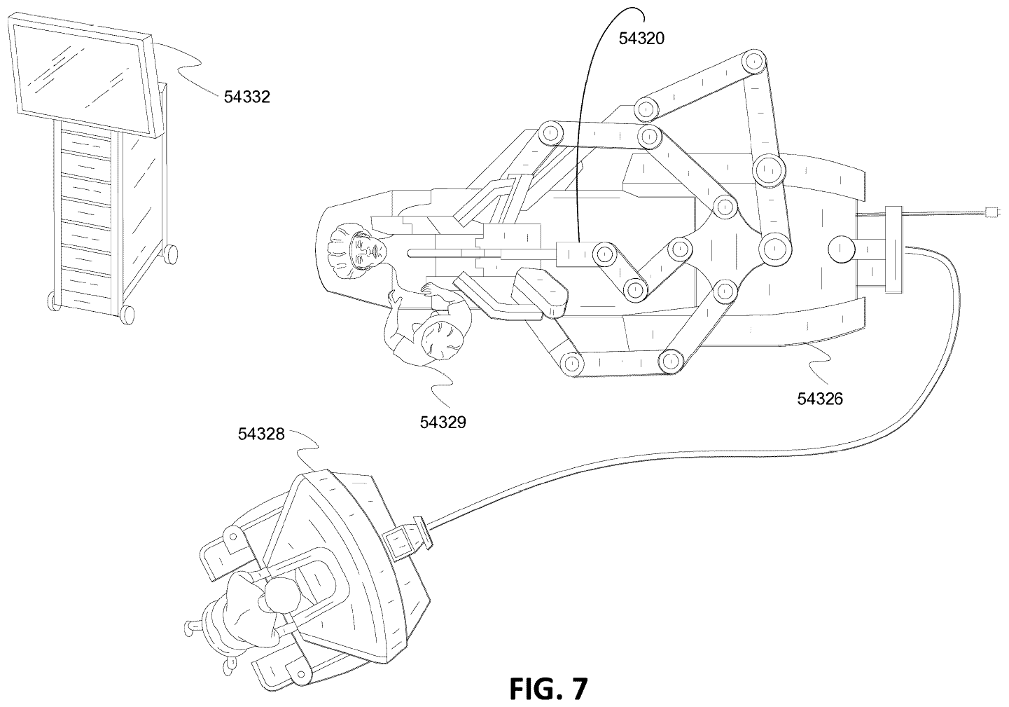

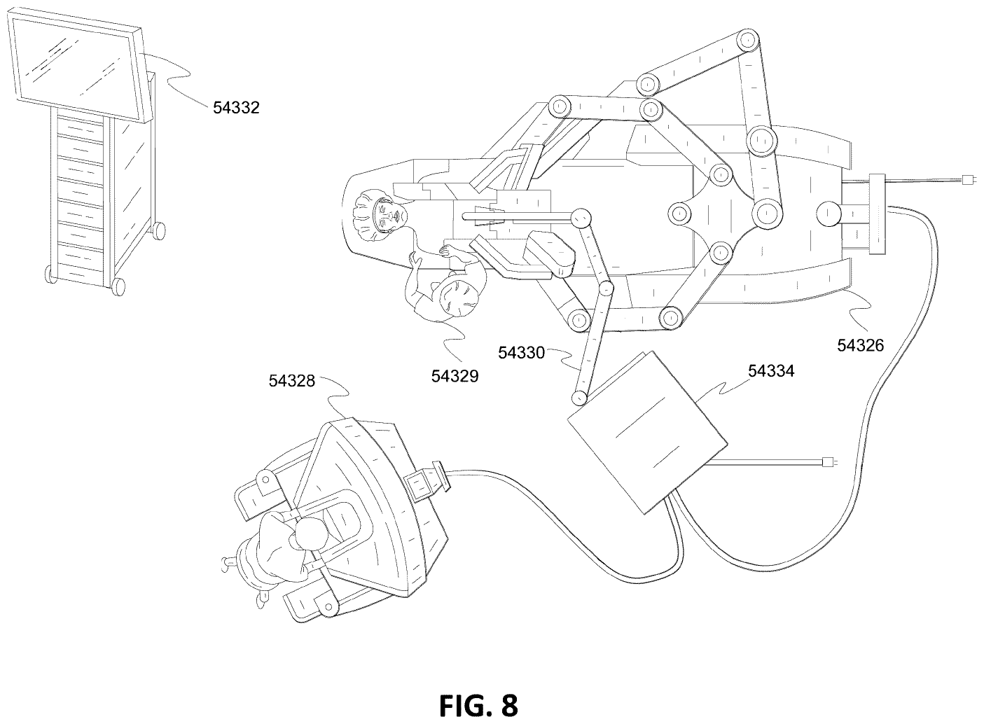

FIG. 7 and FIG. 8 illustrate a stapler device (e.g., a linear or circular stapler) that may be controlled by an HCP together with the main laparoscopic robotic surgical system using a robotic surgical system console.

FIG. 7 illustrates an example where a robotic system with a linear stapler may be configured together with a robotic system with a circular stapler and a smart hub 54332 for performing a robotic colorectal surgical procedure. As illustrated in FIG. 7, the circular stapler may be attached to one of the robotic arms 54320 of a multi-arm robotic surgical system 54326.

In another example, as illustrated in FIG. 8, the circular stapler may be independent and not attached to one of the arms of the multi-arm robotic surgical system. As illustrated in FIG. 8, the circular stapler may be attached to an arm 54330 of a different robotic surgical system 54334 (e.g., a single arm robotic surgical system). The main robotic surgical system 54326 may communicate with the secondary robotic surgical system 54334 via a wired or wireless interface (e.g., via a Wi-Fi interface or a Bluetooth Low Energy (BLE) interface).

As illustrated in FIG. 7, where the circular stapler is attached to one of the arms 54320 of a multi-arm robotic system 54326, which is connected to the main console 54328. A lead HCP (e.g., the main surgeon heading the surgical procedure) may sit behind the main console 54328 to direct and control the robotic arms and operate the surgical instruments attached to the robotic arms of the multi-arm robotic system 54326. The lead HCP may operate the circular stapler with help of the second HCP 54329 who may be in proximity to the patient and the circular stapler itself.

In either of the setups illustrated in FIG. 7 and FIG. 8, the HCP operating the main multi-arm robot may begin the surgical procedure by laparoscopically dissecting the area surrounding the colon of the patient. The HCP using various surgical instruments attached to the laparoscopic robotic surgical system may transect the colon with a linear stapler or a circular stapler and remove the damaged areas, as described herein.

In case of FIG. 8, where the circular stapler is attached to an independent robotic surgical system 54334, the HCP operating the primary robotic surgical system 54326 may request control of the circular stapler from the secondary robotic surgical system 54334. Once the lead HCP operating the primary robotic system establishes control with the secondary robotic surgical system and the circular stapler, the lead HCP at the console 54328 of the primary robotic surgical system may be presented with controls that are specific to the secondary robotic surgical system 54334 and the surgical instruments attached to the secondary robotic surgical system, for example, anvil closure, firing, opening, etc. A second HCP 54329 (e.g., a resident HCP) may be in proximity of the secondary robotic surgical system 54334 and may be in physical control of the circular stapler attached to the secondary robotic surgical system 54334.

In either of the setups illustrated in FIG. 7 and FIG. 8, the lead HCP may instruct the second HCP 54329 about physical placement of the stapler. The lead HCP may instruct the second HCP 54329 about initial placement of the stapler with respect to the surgical site. The lead HCP may provide further instructions to the second HCP 54329 about the next steps that are to be performed in the surgical procedure. Under the lead HCP's direction, the second HCP 54329 may perform one or more of the following steps: position a circular stapler or device at a desired position; insert the anvil; or attach the anvil to the trocar of the circular stappling surgical instrument. The circular stappling surgical instrument may now be prepared for a circular firing. The lead HCP may be presented on the main console controls associated with the primary laparoscopic multi-arm robotic surgical system, which may be overlayed with the controls associated with the circular stapler attached to the secondary robotic surgical system. The lead HCP may send commands to the secondary robotic surgical system or the smart surgical system attached to one of the arms of the secondary robotic surgical system using the wired or wireless communication path established between the console and the secondary robotic surgical system or circular stapler.

In either of the setups illustrated in FIG. 7 and FIG. 8, the lead HCP may send a command to the circular stapler (either directly or via the secondary robotic surgical system) for beginning of the closure. In addition to the lead HCP sending the command to the secondary robotic surgical system or the circular stappling device, the second HCP may monitor the mechanical aspects of the closure (e.g., tissue compression scale, etc.) The circular stappling device may communicate an indication to the main console indicating that appropriate compression value has been reached and the stappling device closure stroke is stopped and held at the compression reading. In addition, the second HCP may visually verify the compression is within an acceptable zone (e.g., green zone) of the tissue compression scale. The lead HCP may use its console 54328 to visually verify that the two tissue ends are captured correctly and circular stapler is ready for the firing step. In FIG. 8, where the circular stapler is part of a different robotic system than the one the lead HCP is directly controlling, the lead HCP on its console 54328 may have access to an imaging stream being shared by the single arm robotic surgical system 54334 that is housing the surgical stapler. Using the shared imaging stream, the lead HCP may also visually verify that the two tissue ends are captured correctly and circular stappling device is ready for the firing step.

Once the readiness of the circular stappling device is verified, the lead HCP (either directly/locally or remotely) may send commands to the circular stapler to fire. Once firing of the circular stapler has been completed, the lead HCP may command the circular stapler to open the anvil. The lead HCP may then relinquish control of the circular allowing the second HCP 54329 to remove the circular stapler.

The position of the circular stapler may be determined by attaching it to a robotic arm, as illustrated in FIG. 7 and FIG. 8. As illustrated in FIG. 7, the robotic arm may be part of the primary robotic surgical system (e.g., the main laparoscopic robotic surgical system). As illustrated in FIG. 8, the circular stapler may be attached to a separate robotic surgical system, for example, a single arm robotic surgical system 54334 or a docked support arm. This may enable the lead HCP sitting at the main console 54328 to indicate that care may be needed to prevent excessive pressure to the linear stapple line while also confirming when the circular stapler distal head is within an acceptable range.

Systems, methods, and instrumentalities described herein may allow more than one independent surgical systems (e.g., an endoscopic robotic system and a laparoscopic robotic system) to act on same input data by processing two independent control requests (e.g., two independent and/or parallel direct user control requests). The two independent surgical systems may operate as if the input data was provided directly to each of the systems independently. Such an arrangement may allow the main console to send commands including control requests to each of the systems that may behave as independent surgical systems, yet perform coupled or linked operations.

In an example, more than one remote user requests may be synchronized. The synchronization of the remote user requests may originate from a common console that may have capability of generating the remote user requests using more than one protocols that are compatible with the surgical systems being controlled by the console. The commands used for user requests may be sent using wired, wireless, or other interfaces, for example, using a user input/output device (e.g., a touchscreen, keyboard, mouse, joystick, etc.).

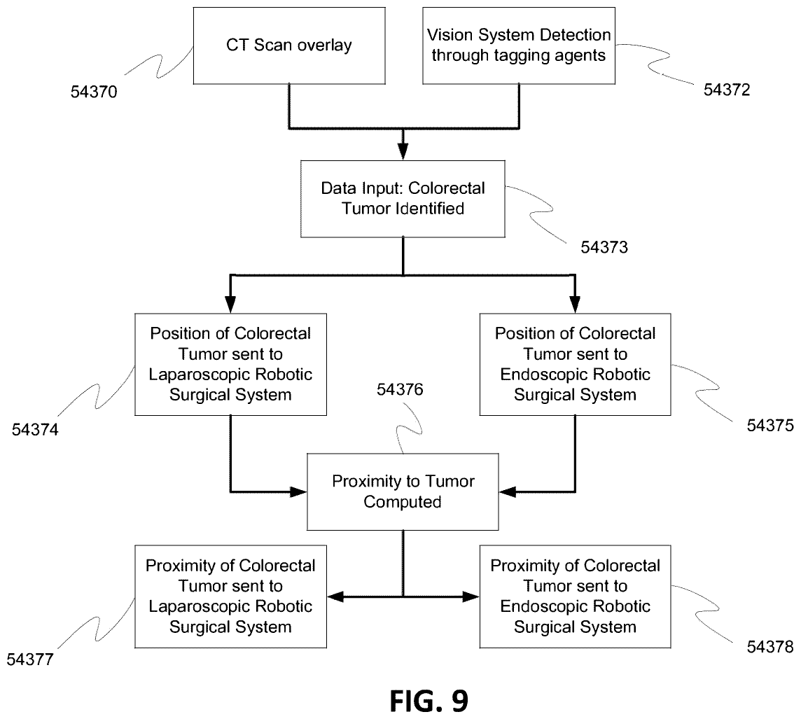

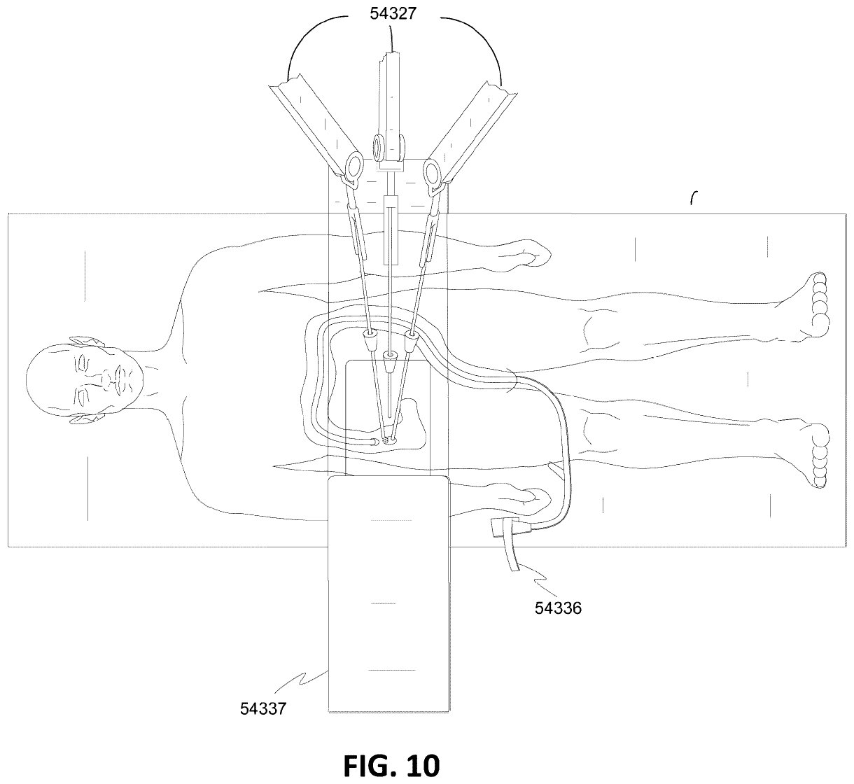

FIG. 9 illustrates performing a pre-surgical procedure for identifying a cancerous tumor, which is followed by performing a colorectal surgical procedure to remove the identified cancerous tumor. As illustrated in FIG. 9, at 54370, a pre-surgical procedure (e.g., a CT scan 54337 as illustrated in FIG. 10) may be performed on a patient for identifying a tumor that may require removal. At 54372, vision system detection through tagging agents may be performed. At 54373 a combination of CT scan overlay and vision system detection through tagging agents may be utilized to identify a polyp or a cancerous colorectal tumor. Once the colorectal tumor is identified, at 54374, position of the colorectal tumor with respect to the laparoscopic robotic system may be determined. At 54375, position of the colorectal tumor with respect to the endoscopic robotic system may be determined. At 54376, proximity of each of the laparoscopic robotic system and the endoscopic robotic system may be computed (e.g., computer together) and sent independently to the two robotic systems. At 54377, the proximity of the colorectal tumor to the laparoscopic robotic system may be sent to the laparoscopic robotic system. At 54378, the proximity of the colorectal tumor to the endoscopic robotic system may be sent to the endoscopic robotic system.

Once the presence of the colorectal tumor is identified, and the proximity of the colorectal tumor to each of the robotic systems is computed and communicated (communicated separately) to each of the robotic surgical systems, each of the two independent robotic surgical systems may operate in a linked fashion during the surgical procedure, as illustrated in FIG. 10.

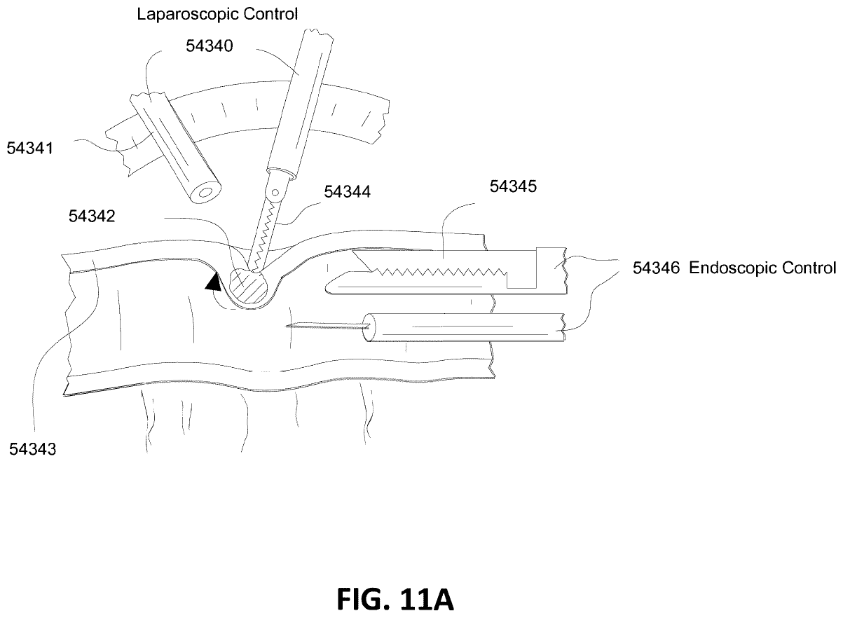

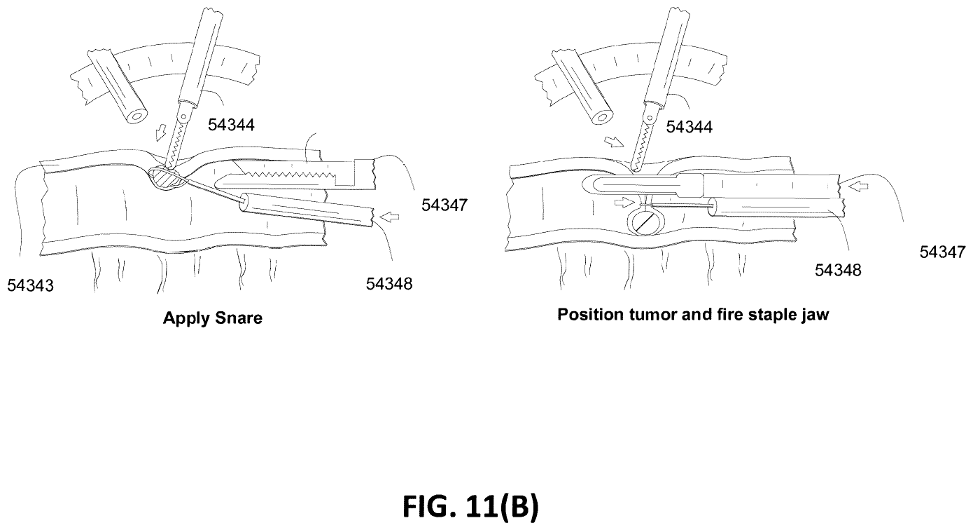

As illustrated in FIG. 10, laparoscopic robot arms 54327 that are part of a multi-arm robotic surgical system and an endocutter in combination with a scope 54336, which is a part of an endoscopic robotic surgical system may be utilized to perform a colorectal surgical procedure, as described herein. As described in FIGS. 11A-11B, the endoscopic robotic system may be utilized to snare and fold the tissue wall pinching in closed and the endocutter can then be placed over the fold separating the endoscopic grasped tissue and sealing the hole that is created in the side. During the stapler firing step of the robotic surgical system controlling the endocutter, it may operate in tandem robotic surgical system controlling the endoscope such that the endoscopic retraction may be applied to pull inward as the stapler is pulled outward. Both the commands for executing the staple firing step and the endoscopic retraction step may be issued in parallel to the endocutter device (which may a part of the multi-arm laparoscopic robotic surgical system) and the endoscopic device (which may be a part of a separate independent robotic surgical system). In an example, the commands may be sent to the two robotic surgical systems (e.g., sent from a main console controlling the two independent robotic surgical systems) without the two robotic surgical systems exchanging information directly.

FIGS. 11A and 11B illustrate an exemplary colorectal tumor removal surgical procedure using techniques described herein. As illustrated in FIGS. 11A and 11B, once the presence of the colorectal tumor is identified using the CT scan device 54337 of FIG. 10, endoscopic robotic surgical system and laparoscopic robotic surgical system may be utilized to perform the surgical procedure.

During the actions illustrated in FIG. 11A and FIG. 11B, a lead HCP may control the laparoscopic robotic surgical system 54340 including the grasper 54344 and scope 54341. A second HCP may control the endoscopic surgical system 54346 including the stapler 54347 and the snare 54348. As illustrated in FIG. 11A, the lead HCP may send commands to position the grasper 54344 next to the tumor 54342 against the colon serosal layer 54343, and the second HCP, using endoscopic control 54346 may position the stapler 54347 and the snare 54348 in position next to the tumor 54342.

As illustrated in FIG. 11B, the lead HCP may send command to the laparoscopic grasper 54344 to push the tumor 54342 further into the colon (e.g., toward the endoscopic devices). The second HCP may send commands to endoscopic surgical system for applying the endoscopic snare 54348 to the inverted tumor 54342. The second HCP may then send commands to retract the snare to pull the tumor further into the colon. The movements of the stapler and the snare are shown by arrows next to the stapler 54347 and the snare 54348. The lead HCP may continue to push the tumor into the colon while the second HCP may fire an endoscopic stapler 54347 to separate the tumor from the colon tissue and staple the resulting incision. Commands may be sent independently to the laparoscopic robotic surgical system to push the tumor into the colon and to the endoscopic surgical system for stapler 54347 stappling and moving in the forward direction and for the snare 54348 to move in the opposite direction. The commands to the two surgical systems may be sent independently.

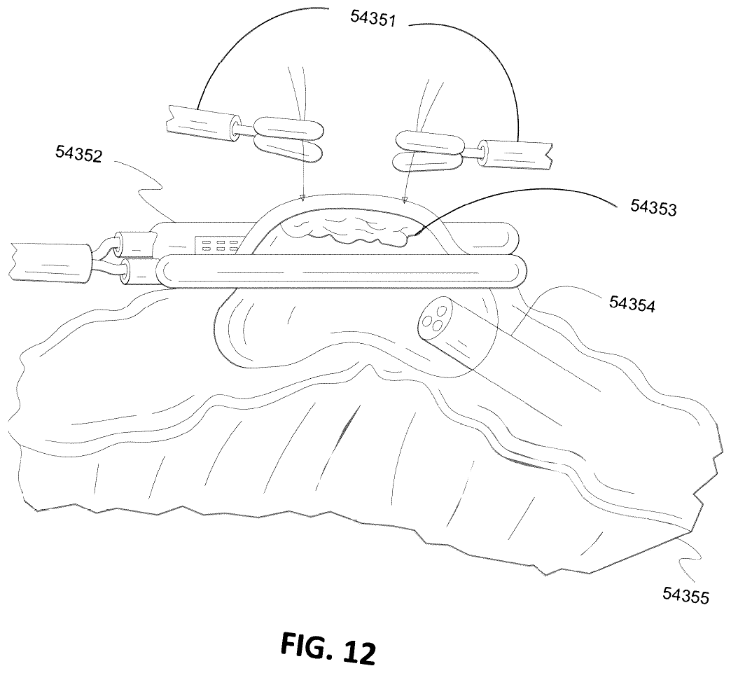

FIG. 12 illustrates an example tumor removal using techniques described herein. Colonoscopy-assisted wedge resection technique may be used for polyps located near the antimesenteric side of the colon. Laparoscopic wedge resection with intraoperative colonoscopy may be performed for removal of large tumors that may not be treated endoscopically.

As illustrated in FIG. 12, colonoscopy assisted laparoscopic edge resection (CAL-WR) surgical procedure may involve one HCP operating and controlling the laparoscopic robotic surgical system and the other HCP operating and controlling the colonoscopy robotic surgical system.

CAL-WR surgical procedure is initiated with the lead HCP performing diagnostic laparoscopy with the insertion of multiple trocars. The spot of the tumor 54353 in the colon 54355 may be identified and the corresponding part of the colon may be mobilized. Mobilization may be performed to enable the HCP the ability of placing the stapler 54352 (which may be part of the laparoscopic robotic surgical system) in the best possible position.

The second HCP may mobilize the endoscopic scope and place it next to the tumor site. The lead surgeon may laparoscopically place a suture near the tumor with intraluminal endoscopic visualization. Traction may be provided on the suture to enable positioning of the stapler 54352. The lead HCP may then send commands to fire the stapler 54352, which is part of the laparoscopic robotic surgical system and confirm total inclusion of the tumor 54353 using the endoscopic robotic surgical system 54354. The two commands may be sent in parallel without the laparoscopic robotic surgical system and the endoscopic robotic surgical system interacting with each other.

In an example, multiple surgical systems or robotic surgical systems may operate together in performing steps of a surgical procedure. In addition, the surgical systems may all be from different manufacturers and may have different control systems. Any of the surgical systems, therefore, may not be configured in a manner to surrender full control of its operation to another manufacturer's surgical system. Such an arrangement may be prohibited, for example, one or more of the following reasons: patient safety, one surgical system may not have a full understanding of operation of a second surgical system, nor the willingness to assume accountability for its safe operation, loss of the propitiatory data recorded or the operational program. However, in case of an integrated operating room, the surgical systems may operate independently as originally designed and certified but with an ability to accept requested commands on operation from an HCP via an intermediate smart system.

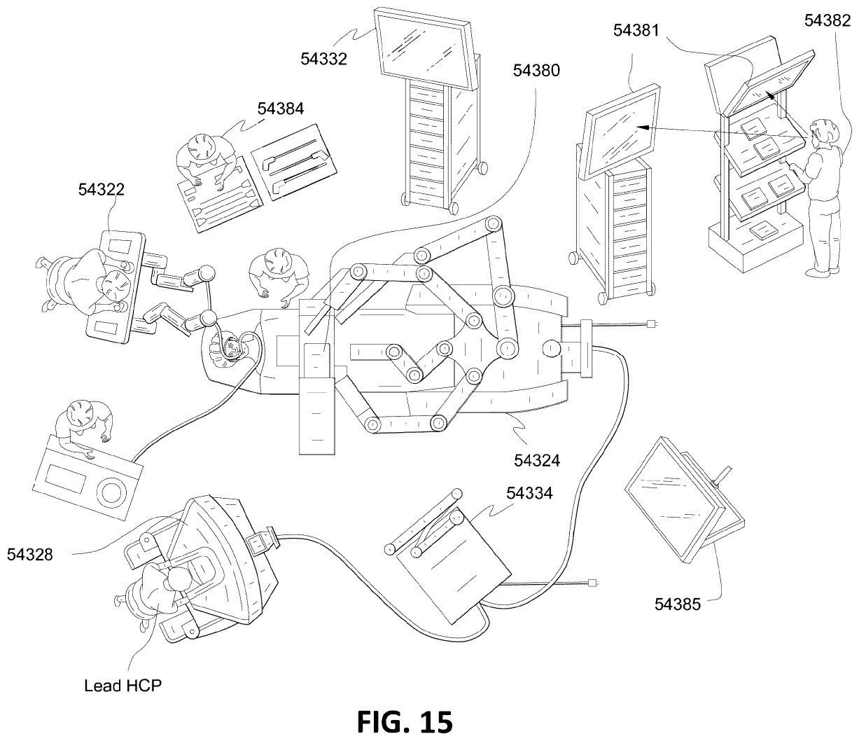

In an example, external imaging system (e.g., a cone-beam CT) may operate with another smart system and may need to be repositioned or the image focal location or orientation may need adjusting. In an example, for example in a thoracic surgical procedure, an imaging system may be used in cooperation with a flexible endoscopy surgical device (e.g., a hand-held endoscopy surgical device or a robotic endoscopy surgical device). A flexible scope may be extended further into the bronchus. Such extension of the flexible scope further into the bronchus may then need the imaging system is to adjust its orientation (for example, as illustrated in FIG. 15) to keep the distal tip of the flexible scope in the field-of-view or even centered in the field-of view. In such a case, the imaging system may receive a request from an HCP sitting at the main console of the flexible scope or an intermediate smart system to adjust the imaging system.

In another example, the imaging system may be automatically adjusted based on the relative position of the flexible scope in the bronchus of the patient. In such an arrangement, manner where the surgical system controlling the flexible scope or the flexible scope itself may provide updates regarding the scope position information to the imaging system and the imaging system may utilize the updates regarding the scope position information to adjust its position accordingly. The updates regarding the scope position and operation information may include information about the operation, position, and adjustments of the position of the flexible scope.

In an example, the flexible scope, as part of preemptive alignment, may instruct the imaging device about the timing of movements and locations and/or directions associated with the movements (e.g., as illustrated in FIG. 15). The preemptive alignment may be applied proactively. For example, the imaging system may be instructed to move to a position tracking a specific part of the bronchus and then the flexible scope would be moved to that position in the bronchus.

In an example, a first smart surgical system may have discernment of its limitation relating to the actions of a second smart surgical system. This decision may result in the first smart surgical system requesting the second surgical smart system for operational instructions in remote controlled fashion.

In an example, an originating smart surgical system may determine that one or more actions to be performed as part of a surgical procedure are outside of its physical or processing capabilities. The originating smart system may discover and/or reach out to nearby neighboring smart surgical system for assistance. The originating smart surgical system may prepare to surrender control to the neighboring smart system that may have the capability of supporting one or more actions. In an example, neighboring smart system may yield itself and request alternate smart surgical system to perform action requested by the originating smart system.

Systems, methods, and instrumentalities are described herein for enabling full remote control of one smart surgical system by another smart surgical system. For example, in case of using robotic flexible endoscopy used with robotic laparoscopy, the laparoscopic robot console may be configured (e.g., may assume) as the primary robotic surgical system and the robotic surgical system controlling the flexible endoscopy unit may configured as another minion of the console just like the laparoscopic robotic arms of the laparoscopic robot system. In an example, a robotic surgical system (e.g., a Hugo robotic system) may have multiple minion independent cart arms tethered to a control console. In this case the flexible endoscopy robot may be attached to either the main tower or directly to the robot control console allowing the controls to interactively control all of the attached arms interchangeably.

Features described herein may allow a primary surgical system or a primary robotic surgical system to have direct integrated control of a minion surgical system. In an example, operating mode of a minion system may be the same (e.g., from the same manufacturer and operating on a compatible version of software) as that of a primary robotic surgical system. In such a case, the minion system may be integrated and/or attached to an arm of the primary robotic surgical system. The minion surgical system attached to one of the arms of the primary robotic surgical system may be inter selectable (e.g., like any of the other arms of the robotic surgical system). The minion surgical system may be controlled using the common interface or the main console of the primary robotic surgical system.

In an example, a dual visualization comprising the primary robotic system and the minion system may be presented on a common interface or the main console connected with the primary robotic system. For example, a dual visualization may be presented using one of the following: a picture-in-picture mechanism, it or an integrated mechanism, for example, using overlays or transparent views that may merge the imaging associated with the two surgical systems enabling an HCP to see through or behind tissues and/or organs. In examples, merging or overlaying may include using augmented reality to add or overlay imaging associated with one surgical system over the imaging associated with the other surgical system. In an example, the user interface or the main console display showing the HCP what they normally expect from a real-time visual light image of the surgical site while then being able to add or supplement portions of the view that the secondary imaging could add data about.

Features described herein may allow more than one surgical systems (e.g., a primary surgical system and a minion surgical system) to operate in tandem in a primary-minion mode (e.g., even if the two surgical systems may not be compatible to be integrated directly). In such an arrangement, an imaging stream (e.g., a video stream) may be ported from the minion surgical system to the user interface or main console that may be a part of the primary surgical system. In addition, the primary surgical system may be used as controller for controlling various aspects of the minion surgical system. In an example, the primary surgical system may send control signals and/or commands to the minion surgical system. The controls for effecting movement on the minion surgical system may be simulated or emulated by the primary surgical system allowing it to be an I/O system for the minion surgical system.

In example, multiple minion surgical systems may be controlled (e.g., simultaneously controlled) by a primary surgical system. A surgical system with integrated with a scope imaging system (e.g., Olympus EBUS scope) and a flexible endoscope may be configured as minion surgical systems that may be controlled by a primary surgical system (e.g., a Hugo laparoscopic robotic surgical system). In case of the primary-minion control model, the primary surgical system may autonomously establish partial control of the minion system(s).

In an example of removing gallbladder stones surgical procedure below, one surgical system (e.g., Hugo robot) may be configured and/or positioned as the primary robotic surgical system, for example, to perform cholecystectomy. The primary robotic surgical system may be used as the primary robot for imaging and as the main visualization source and main control console interface to be used by one of the HCPs (e.g., the surgeon) involved in the surgical procedure. Another surgical system (e.g., a Monarch flexible robotic system) may be used for controlling the endoscope portion of a scope imaging system (e.g., Olympus EBUS ultrasound secondary scope) for imaging of the stones and ducts. The ultrasound image from the scope may be overlaid on display of the primary surgical system (e.g., Hugo system) to visualize the underlining structures from the laparoscopic side. For example, the HCP controlling the primary surgical system may redirect the scope slightly to get a better image. The HCP may have direct control of the primary surgical system as well as requested, but independent, control of the scope imaging system.

In an example, and in addition, to obtain the desired imaging view using the scope imaging system, it may be reoriented (e.g., slightly reoriented) such that the primary surgical system may request the minion surgical system to adjust the control cables of the flexible scope such that the head location may allow the scope imaging system to have a better view. The request may be sent (e.g., autonomously sent) by the control system of the primary surgical system control system (e.g., without any intervention of an HCP). The request may be sent by the primary surgical system, for example, because the HCP was busy controlling the scope imaging system and a physical movement of the scope was need in addition to the control adjustments of the scope imaging. In such an example, the primary surgical system and the HCP may supply I/O interface data to a specific minion system, which in turn may operate as expected. In this case, the primary surgical system may direct the minion system(s) without taking control of the minion system(s).

Features described herein may provide reversible or bi-direction primary-minion control exchange. In this case, an HCP's interaction with various surgical systems may be used to identify the primary surgical system. For example, an HCP may move from one surgical system to another and the operational control of the first surgical system may be transferred with the HCP, as the HCP moves from one surgical system to another. In order to ensure that a primary control system is designated at all times without any interruption, each of the surgical systems may attempt to maintain its designation as a primary surgical system. The HCP presence in combination with authentication of the HCP may be utilized to designate a surgical system as the primary surgical system. In an example, authentication of the HCP used in designation of a primary may be performed by using one of more of the following authentication mechanisms: a digital key or token that may be required by a surgical system to establish primary control.

The surgical systems involved in the bi-direction primary-minion control exchange may be aware of each other and the control interface established for the HCP, for example, to track the HCP. In an example, the control interface established for the HCP may include a physical aspect, e.g., a switch, a digital identification, or a biometric monitoring system. In an example, a smart Hub (e.g., a separate Hub) from any of the other surgical systems may be untilted to maintain control and access grants. The smart Hub may inform the surgical systems about the identification of the primary surgical system. The system based on the smart Hub may track the HCPs as they move between surgical systems to granting primary control to the surgical system with which the HCP may be directly interfacing and revoking the primary control when the HCP is no longer interacting with the surgical system.

Features described herein may be provided for dual generator control with both generators existing within the same Hub tower or rack. In an example, operations in combo energy devices such as monopolar-bipolar, bipolar-ultrasonic, or monopolar-ultrasonic energy may be combined. The operations may be combined at the same time or in a serial fashion. In such a case, two separate generators may work in tandem to provide the handpiece or a robotic surgical device the proper energy modality, power level, and/or communicate pressure needs in order to provide the advanced outcome desired. If the two generators are in the same module or in the same smart hub, one of the energy devices may receive commands from the other energy device or both the energy devices may take commands from a primary command source to coordinate their outputs.

Features described herein may be provided for independent smart system evaluation and determination of other system's controllability. In an example, one surgical system (e.g., surgical system A) may request control of the other surgical system (e.g., surgical system B).

A central arbitrator may be required to coordinate the transfer of control. The arbitrator may determine that the first surgical system has the required hardware and/or software to complete the full control transfer. If the arbitrator deems the appropriate level, it may allow for establishment of a direct high speed data/control transfer between the first surgical system and the second surgical system. If the arbitrator determines that full control is not within the capabilities of either the first surgical system or the second surgical system, it may generate an alert indicating the level of control that may be allowed and an indication whether this level of control will be sufficient for the upcoming surgical procedure steps.

A system may be configured with a default level of control which may be the highest degree of control allowed based on the setup of the two systems.

If the arbitrator determines that one surgical system has limitations that may compromise the direct control of the other system, the surgical systems involved and/or the arbitrator may determine if the risk of completing the actions is acceptable.

The final risk determination may cause the surgical system to lower the level of control one surgical system may have over the other surgical system. The determination may be based on the level of control the one of the surgical systems (e.g., the second surgical system) may be able to achieve and the properties and/or requirements of the upcoming surgical steps in a surgical procedure. In an example, higher levels of risk may cause the surgical systems to lower the level of control.

Each of the surgical systems involved in establishing the controllability may acknowledge the request for control to the arbitrator, and each of the three systems may agree on the transfer before proceeding.

Features described herein may be provided for arbitrator master control of multiple surgical systems. The arbitrator may act as the final decision maker as to the level or mode of cooperation between the more than one surgical systems. In addition, the arbitrator may make lower level decisions regarding whether the surgical systems are going to share a new temporary memory stack the systems share.

Features described herein may be provided for establishing shared memory and stack. Each of the surgical systems associated with this new network may utilize the shared memory and/or the stack to process the code and storage areas. Surgical systems may share the shared memory, which may allow up to date access and any modifications that may be needed to memory and/or task control.

The arbitrator may decide whether an additional high speed data line needs to be established between the cooperating systems. If the steps of the procedure require it, the arbitrator may set up this structure and then monitor (e.g., continuously monitors) the procedure, for example, as a safety mechanism.

Features described herein may be provided for a shared full control of one robotic surgical system with another. One of the robotic surgical systems may be designated as a primary surgical system and the second system may also be a primary surgical system. The second robotic surgical system may then allow the first robotic surgical system authority over at least some of the operational characteristics (e.g., not all the operational characteristics) of the second surgical system. The sub-primary robotic surgical system may retain control of all of the aspects of the coupled robotic surgical system, but may allow the primary robotic surgical system to request limited aspects of the sub-primary control.

The sub-primary robotic surgical system may monitor the remotely controlled systems providing them additional, supplementary or override control of the remote-controlled sections. In an example, a Monarch flexible robot may be designated as a primary robotic surgical system and an Otava robot may be designated as the sub-primary robotic surgical system. The sub-primary robotic surgical system may grant remote control of two of its four arms to the primary robotic surgical system for cooperative interaction, for example, while performing an Endo-Lap surgical procedure. The sub-primary robotic surgical system may also provide supplementary control of two remote controlled arms to provide interaction control of the portions of the arm outside the body relative to the patient, the table and the other two arms. An HCP using the primary robotic surgical system may move the two remote arms inside the patient, the sub-primary robotic surgical system may provide some direction to the joints outside the patient for both the robotic surgical systems to orchestrate their movement to prevent collisions outside the patient's body while the HCP is controlling the end-effector motions inside the patient's body.

In an example, supplementary surgical system modules may also establish primary and sub-primary relationship and operate in concert. Advanced energy generator, the advanced visualization modules, smoke evacuators or patient critical modules like insufflation or ventilation system maintaining their prime operational directives and the other systems allowed to interface with some control aspects unless those aspects interfere with their primary operational mode.

In an example, a smart ventilation system may be shared sub-primary controlled by a smart hub or a robot hub. For example, the ventilation system may allow the smart hub or the robot hub to vary the ventilation rate and the oxygen levels as long as they stay within a preconfigured and pre-set patient operation envelope. The smart hub or the robot hub may also operate other controls of ventilation system, including for example, air volume, air pressure, etc. to keep functioning as intended. If the remote control from the smart hub or the robot hub drives a controlled factor of the ventilation system to a point where the ventilation system is being pushed out of its normal operating mode, or one or more of the patient biomarker measurements indicate a critical situation then the sub-primary surgical system may regain full primary control of its system to re-balance the settings based on its primary control algorithms. In this case, the sub-primary surgical system may notify an HCP (e.g., an HCP on a remote system or the primary surgical system) the reason for the sub-primary surgical system taking back full control and/or rejecting a request the sub-primary surgical system may have received from the primary system. The sub-primary surgical system may allow for the HCP to control the sub-primary surgical system within this marginal range, but may prevent it from moving anything to critical or dangerous range.

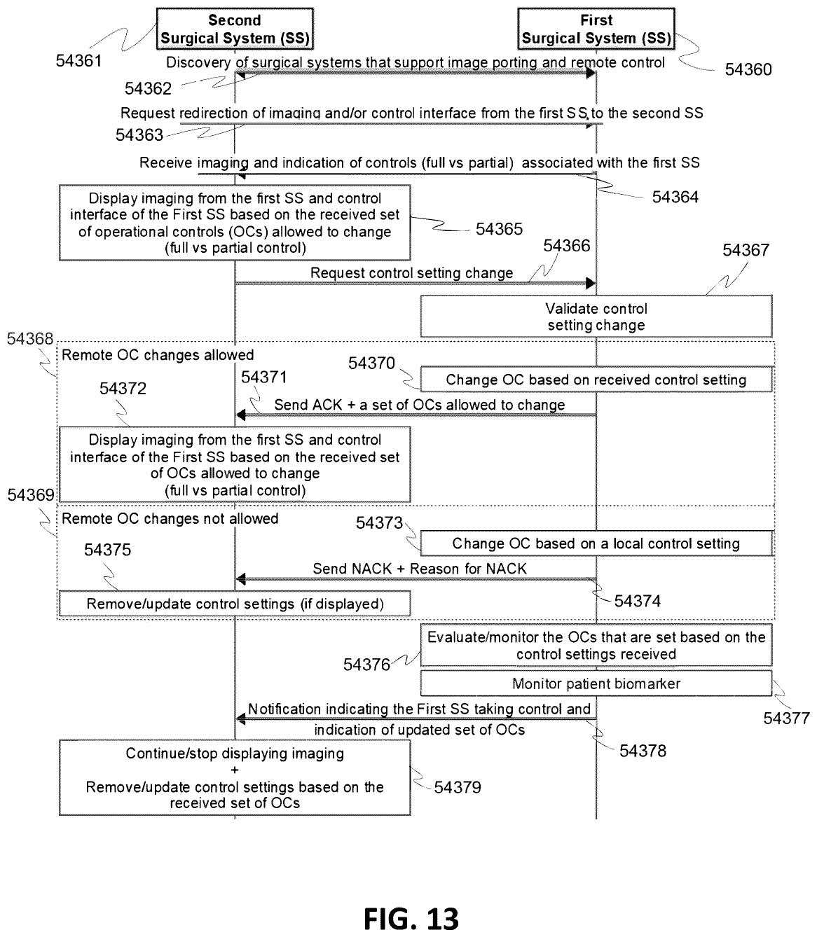

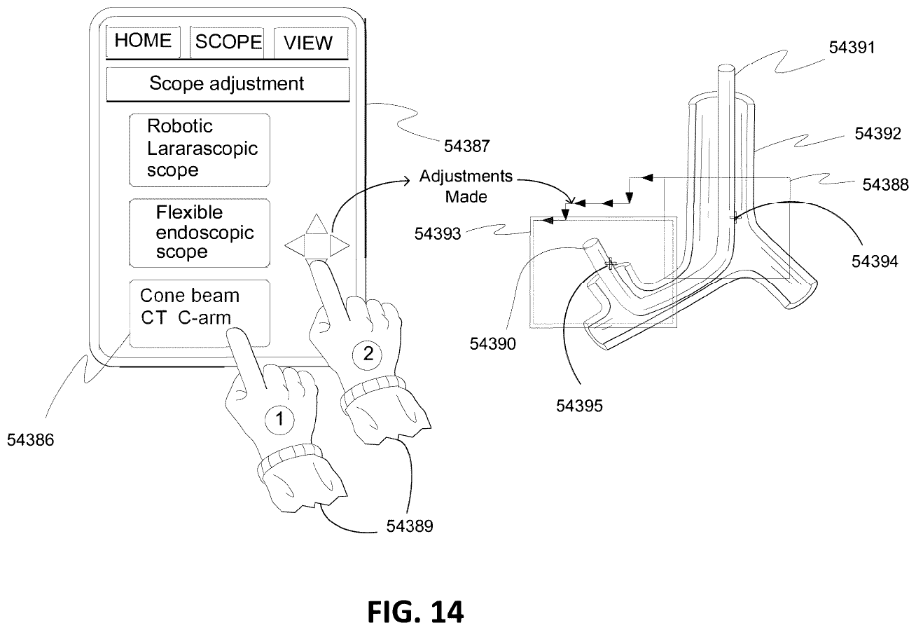

FIG. 13 is a message sequence diagram illustrating control sharing between two surgical systems (first surgical system 54360 and second surgical system 54361) used in a surgical procedure. The surgical procedure may include a colorectal surgical procedure (as illustrated and described herein in FIGS. 7 through 10, 11A, 11B, and 12) or a thoracic surgical procedure (as illustrated and described herein in FIG. 6, FIG. 14, and FIG. 15), the first surgical system may be a endoscopic robotic surgical system that may be autonomous or operated by one HCP and the second surgical system may be an laparoscopic robotic surgical system that may be operated by another HCP (e.g., the lead HCP).

As illustrated in FIG. 13, at 54362 discovery of surgical systems that support image porting and remote control may be performed (e.g., by smart surgical systems in an operating room, with or without a smart hub). Based on the discovery information, one or both of the first surgical system 54360 and the second surgical system 54361 supports image porting and remote control.

At 54363, the first surgical system 54360 may receive a request (e.g., second surgical system 54361 may send a request) associated with redirection of imaging and/or a control interface from the first surgical system 54360 to the second surgical system 54361 (e.g., remote control surgical system).

At 54364, the second surgical system 54361 (e.g., based on the request) may receive imaging and indication of controls (e.g., full control or partial control) associated with the first surgical system 54360. At 54365, the second surgical system 54361 may display imaging from the first surgical system 54360 and the control interface of the first surgical system (e.g., based on a received set of operational controls (OCs) that the second surgical system 54361 is permitted/enabled to change/modify). The imaging received from the from the first surgical system 54360 may be added to the display of the second surgical system 54361.

At 54366, the second surgical system 54361 may request a control setting change based on the set of OCs received from the first surgical system 54360. The first surgical system 54360 may determine whether to validate the control setting change. At 54367, the first surgical system 54360 may validate the control setting change. In case the validation of the control setting change is successful, at 54368 (i.e., remote operational control changes are allowed), at 54370, the first surgical system 54360 may change the control setting (e.g., a set of OCs) based on the received control settings from the second surgical system. At 54371, the first surgical system 54360 may send an acknowledgment to the second surgical system 54361 indicating the control setting change. The first surgical system 54360 may send an additional (e.g., updated) set of OCs that the second surgical system is enabled (e.g., permitted) to change.

At 54372, the second surgical system 54361 may display updated imaging from the first surgical system and an updated control interface of the first surgical system based on the received set of OCs that it is permitted to change.

In case the validation of the control setting change is not successful, at 54369 (i.e., remote operational control changes are not allowed), At 54373, the first surgical system 54360 may determine to reject the requested control setting change and change the OC based on local settings instead. At 54374, the first surgical system 54360 may send a negative acknowledgement (NACK) and/or a reason for NACK to the second surgical system 54361. At 54375, the second surgical system 54361 may update (e.g., remove) control settings based on the received NACK. The second surgical system 54361, based on the received NACK may determine to terminate remote control of the first surgical system by the second surgical system.

At 54376, the first surgical system 54360 may evaluate and/or monitor OCs that are set based on the control settings. At 54377, the first surgical system 54360 may monitor data (e.g., patient biomarker data), to determine control settings. Based on monitored data associated with a patient, the first surgical system 54360 may determine that the patient biomarker value has crossed a threshold value. The threshold value may be preconfigured or negotiated between the first surgical system 54360 and the second surgical system 54361. Based on the determination that the patient biomarker value has crossed a threshold value, the first surgical system 54360 may terminate remote control. At 54378, the first surgical system may send a notification indicating that termination of the remote control and/or the reason for termination of the remote control and that the first surgical system is assuming the control. At 54379, the second surgical system 54361 may update (e.g., remove) the control settings and/or imaging based on the received notification.

Features described herein may be provided for dual generator cooperative operation of combo devices that may have more than one generators (e.g., in separate hub towers or racks). For example, in case of two cooperative generators that may be configured for a single combo device in separate control or communication hubs, one of the cooperative generators may be designated as the primary system. The cooperative generator designated as the primary system may receive inputs (e.g., control inputs) from an HCP for controlling the main energy modality. The primary system may then request the second generator (e.g., the sub-primary system) to supply its energy when and how it may be needed to complement the primary system's energy. The non-primary generator may run its energy generator's main operation and safety aspects as normal, and may consider the shared control commands it may receive from the primary generator as instructions about where, when, and how to provide the supplemental combo energy to the primary generator for performing advanced operation of the combo device.

In an example, a uterine manipulator may be attached to a robotic control arm. Use of an uterine manipulator being introduced through an externally control robot arm control. Examples of the use of a robotically controlled uterine manipulator are described in U.S. Pub. No. 2023/0077141, entitled “Robotically controlled uterine manipulator,” filed Sep. 21, 2021, the disclosure of which is incorporated by reference herein. Primary motion of the dissection of the surgical procedure may be controlled from the main console controlling laparoscopic instruments control of a second system. The uterine manipulator can be at the console or at the bedside when at the console the commands to the uterine manipulator are limited to up down left right, providing for presentation of the dissection planes in the laparoscopic view of the balder and rectum respectively. The in/out motion of the uterine manipulator may be limited by the console commands (e.g., not able to be commanded at the console) to prevent perforation of the uterus. The in/out motion of the uterine manipulator may be limited to manual at the bedside via gravity compensated motion of the robotic arm manually moved, with optional geofencing of up down or left right movements.

Features described herein may be provided for multi-user control of a multi device smart system, for example, within a shared environment. In examples, surgical environment, for example, operating rooms may often be configured with more than one robots or robotic surgical systems and/or more than one smart systems along with multiple HCPs. Each of the HCPs and the surgical or smart systems may interface or interact with each other while performing a surgical procedure. Surgical instruments/surgical devices/surgical systems may allow access and/or control of a function by a unique or trusted HCP. However, in a multi-user environment and/or multi-device smart systems may create different challenges, for example, dealing with conflicting task/execution and or changing demands based on user preference. In this case, each smart system may deal with one or more of the following scenarios: when allowing access off of different systems the smart system may only display the usable command or options to a specific HCP based on defined level of controls (e.g., the surgeon may have full control in any situation, unless a senior surgeon overrides the surgeon's command, a nurse may be allowed to reposition a robot but only in safe conditions); negotiation between HCPs to resolve conflicting demands; override human errors; allow for collaboration with other HCPs (e.g., surgeons) either in or outside of the operating environment, which may allow for HCPs or surgeons from anywhere in the world to assist or guide a surgical procedure. The HCP or the surgeon may have credentials to control the commands for operation but not able to move robot location or instruments attached to the robots, which would require a different HCP to complete a task while the HCP or the surgeon may perform other tasks.

Manual or autonomous controls may be provided. For example, a surgical system capable of full control behavior may have the capability of potentially operating on any of the different levels. The surgical system may operate on different levels with different surgical system simultaneously with separate smart systems.

In an example, the most basic mode of operation may be independent by requested mode. In an example, various systems may be from the same manufacturer or may have been designed perform as such, the most comprehensive primary-minion control may be utilized. The shared control may be used as an optional addition to the primary-minion control while the control may be retained by the built-in control system. In this operational state a hierarchical order or control may be provided. The hierarchical order or control may be based on where the primary HCP is located. In an example, the hierarchical order or control may also be based on priority/safety/criticality, or the main controls (e.g., main controls may have primary priority over any remote controls). Verifying the authenticity of data communicated from a surgical instrument to the communication hub is described in U.S. Pub. No. 2019/0200844, entitled “Method of hub communication, processing, storage and display,” filed Dec. 4, 2018, the disclosure of which is incorporated by reference herein.

In examples, at least two HCP consoles from separate robot surgical systems may be utilized for controlling a separate single smart system simultaneously. Smart system may separate control of different actions of device to multiple controllers.

Features described herein may be provide for multi-user control of a single device smart system within a shared environment. Single device may be simultaneously controlled by multiple HCPs, for example, each HCP may utilize unique control methods.

In examples, device location, movement, and/or positioning may be controlled by smart vision system. Device energy activation/staple deployment may be controlled by an HCP (e.g., the lead HCP or a surgeon) or an alternate HCP who may be designated as controller.

FIG. 14 illustrates an example of an HCP 54389 in control of one robotic system being able to control or adjust other robotic surgical systems (e.g., other local surgical systems). As illustrated in FIG. 14, the HCP may be in control of a robotic endoscopic surgical system during a thoracic surgical procedure, for example. After positioning the flexible scope 54391 at the desired location 54390 inside the bronchus 54392, the HCP using the user interface 54387 may select the Cone beam CT C-arm selection 54386 to adjust or move the cone beam CT c-arm from its current position 54388 to a desirable position 54393 in order to correct the focal point of the cone beam from position 1 54394 to position 2 54395. The user interface 54387 may be provided on a fixed console or a mobile device (e.g., a tablet).

In an example, a handheld circular stapler may establish connectivity with the robotic console. The circular stapler may be configured and may be used and/or controlled as part of robotic and laparoscopic surgical procedures.

In an example, a circular stapler may be positioned and held by an HCP (e.g., an assistant to other HCP). The device firing and closure controls may switch back and forth between the HCPs (e.g., between an assistant and a lead surgeon). Operation of a circular stapler may require inserting and controlling by a non-sterile assistant, but stapler may require it is highly desirable for device feedback and control associated with the circular stapler to be provided to the lead surgeon, who is sterile.

A circular stapler with remote connectivity may provide feedback to an HCP or a surgeon operating the main console controlling a robotic surgical system and also control the circular stapler. However, when the circular stapler is to be inserted by one HCP and controlled by the other HCP, the balance and switching of controls may become complex.

A surgical procedure, for example a colorectal surgical procedure may involve a first HCP (e.g., a robotic surgeon) and a second HCP (e.g., an assistant to the robotic surgeon). The first HCP may at the console of the robotic surgical system and may take control of the closure and firing of various system including the circular stapler, for example, when the circular stapler is ready to attach the anvil, close on tissue and ready for firing. Prior to the first HCP being ready for filing the circular stapler, the second HCP may manually insert the circular stapler into the patient. The second HCP may need control of the trocar and anvil in order to safely insert and remove the stapler.

Prior to the insertion process into the body, the second HCP may need to open the anvil fully, remove the anvil, then retract the trocar. These steps may need to be controlled on the device itself, and may be done outside of the surgical field while the first HCP is busy completing other procedure steps. The handheld buttons/controls would need to be active and the console controls be deactivated.

During actual insertion into the body, the second HCP may retain control until the first HCP is ready to extend the trocar and puncture tissue. In an example, extending the trocar may be performed by or under supervision of the first HCP under the first HCP's control. In an example, the first HCP may delegate it to the second HCP who may be instructed to extend the trocar. The second HCP may physically hold the circular stapler and position the rectal stump relative to the end effector.

While the anvil is being installed onto the trocar, no circular device controls may be needed, unless the trocar extension position needs to be adjusted. The adjustment, if needed, would be covered by the first HCP.

When the anvil is fully installed by the first HCP, full device control may be shifted from the first HCP to the second HCP at the console for issuing control commands for closure and firing. After firing, and on removal of the device, the device control may shift back to the second HCP.

If excessive force is noted on the anvil during removal, alerts may be shown to the first HCP at the console, either allowing the first HCP control of opening the anvil further, or prompting the second HCP to open it further. In an example, the circular stapler may automatically adjust itself and user control to both the first HCP and the second HCP are deactivated.

In examples, for a combination harmonic device, one system may be used to control the device positioning and another system may be used to control the device activation. In another example, for a combination harmonic device, one system may be used to control the device positioning, and a second system may be used to control the device RF activation, and a third system may be used to control the device harmonic activation.

In examples, a first robotic surgical system (e.g., an Otava robotic system) with may have a first console that may be controlled by a lead HCP. A second robotic surgical system (e.g., Monarch robotic system) may have a second console that is controlled by another HCP (e.g., an assistant surgeon). Both the surgical systems may interact with each other, for example, to control a single of the second robotic system. A working channel may be autonomously operated from the bending of the flexible scope. One of the HCPs may perform a snaring task while another HCP may perform the positioning of the snare, as described herein.

FIG. 15 illustrates an example of exchangeability of imaging streams and control between various surgical systems and smart surgical systems (e.g., imaging console systems). As illustrated in FIG. 15, various HCPs (e.g., the lead HCP and a assistant HCP) and surgical systems (e.g., robotic surgical systems), smart imaging systems, and a surgical hub may be used to perform a surgical procedure (e.g., a thoracic surgical procedure or a colorectal surgical procedure).

The HCPs involved may include the lead HCP operating the robotic laparoscopic surgical system 54324 using the robotic laparoscopic surgical system console 54328 and/or a single arm robotic surgical system 54334. The lead HCP may also utilize the monitor 54385 that is a part of the robotic laparoscopic surgical system 54324. A second HCP (e.g., an assistant HCP) may operate and control the robotic endoscopic flexible scope 54322. The second HCP may utilize the monitor located above the tower controlling the endoscopic flexible scope 54322. A third HCP (e.g., a radiologist 54382) may operate and control C-arm cone-beam CT system 54380 via the C-arm console and monitor 54381.

In an examples, controls and imaging streams may be shared between the robotic laparoscopic surgical system 54324 and the robotic endoscopic flexible scope 54322. For example, the lead HCP operating and/or controlling the robotic laparoscopic surgical system console 54328 may control the robotic endoscopic flexible scope 54322, for example, to adjust the location of the endoscope to a desired location. In such a scenario, image streaming may be established between the robotic endoscopic flexible scope 54322 and the robotic laparoscopic surgical system 54324 allowing the lead HCP to observe on the robotic laparoscopic surgical system console 54324 what the second HCP may be observing on the monitor located above the tower controlling the robotic endoscopic flexible scope 54322.

In examples, controls and imaging streams may be shared between the robotic endoscopic flexible scope 54322 and the C-arm cone-beam CT system 54380. For example, the HCP operating and/or controlling the robotic endoscopic flexible scope 54322 may require to adjust the focal point of the C-arm cone-beam CT system 54380, as illustrated in FIG. 15. The HCP either using the user interface of the robotic endoscopic flexible scope 54322 or using a separate device, for example, a tablet (as illustrated in FIG. 14) may send commands to the C-arm cone-beam CT system 54380 in order to adjust its focal point such that it is aligned with the desired location of the HCP and the lead HCP. Imaging (e.g., imaging in real time) may be shared between the C-arm cone-beam CT system 54380 and the user interface the HCP is interacting with such the HCP can observe exactly what the HCP controlling the C-arm cone-beam CT system 54380 is observing. In an example, the HCP's user interface may overlay the image generated by the C-arm cone-beam CT system 54380 over that generated by the robotic endoscopic flexible scope 54322.

In an example, the images generated by both the robotic endoscopic flexible scope 54322 and the C-arm cone-beam CT system 54380 may also be streamed to the console of the lead HCP's console may then over lay the image generated by the C-arm cone-beam CT system 54380 over that generated by the robotic endoscopic flexible scope 54322.

In examples, one or more energy devices that include an air suction device, an energy delivery device, etc. one smart system (e.g., a vision system) may be used to monitor the visibility of the area where smoke may be generated and in response the smart system may control the air suction ON/OFF or rate, and a second system may be used to control drug delivery.

Device feedback and algorithms may switch between control systems. For example, when one of the HCPs is positioning a surgical device and/or manipulating a closure system, internal device feedback control of the surgical device may be used to control closure knob or other buttons to limit closure speeds. In an example, this may occur before the surgical device may start communicating with a robotic console.

In an example, when the lead HCP (or surgeon) at the console takes control of closure and firing system, more advanced console based algorithms adjust firing and closure accordingly.

The controls of the device may switch back and forth between HCPs depending on the surgical procedure step, the lead HCP's choice, device feedback, etc. Control switching may be performed manually or automatically. For example, control switching may be performed manually based on user input. The control switching may be performed automatically based on contextual surgical procedure data (surgical procedure step, etc.), or internal device feedback (e.g., load status, etc.).

Some combinations of controls may be active simultaneously for both users. For example, control may be provided to a lead HCP (or surgeon) for firing control, while another HCP (e.g., an assistant HCP) may retain the closure control.

In operating rooms, multiple surgical devices may operate in close proximity to one another. In addition, the devices may all be from different manufacturers and may have different control systems. The devices may not be aware of the presence of other devices. Even if the devices are aware of other devices, the devices may not be able to communicate to coordinate their actions. This lack of coordination may cause the surgical devices to become entangled with each other and, in the worst-case scenario, injure a patient.

A system (e.g., a dual system) may have independent yet simultaneous control of more than one (e.g., two) smart instruments (e.g., by the same user). Feature(s) described herein may provide medical professionals the ability to operate an actuatable instrument from a first robot (e.g., endoscopic working channel tool) at the same time as a second actuatable instrument from a second robot (e.g., a lap powered device). For example, operating multiple actuatable devices at once (e.g., an endoscopic device and a laparoscopic device) may be used to hand off a piece of tissue or anatomy from one to the other.

What is claimed is:

1. A method for operating a first surgical system using a remote second surgical system, the method comprising:
obtaining support and control information associated with remotely operating the first surgical system;
determining, based on the support and control information, that the first surgical system supports image porting and remote control;
requesting, based on the determination that the first surgical system supports image porting and remote control, redirection of imaging and a control interface information from the first surgical system to the second surgical system;
receiving, in response to the request, imaging and control information associated with the first surgical system; and
displaying, based on the imaging and control information, imaging from the first surgical system and displaying a control interface associated with the first surgical system, wherein the imaging from the first surgical system and the control interface associated with the first surgical system is associated with the first surgical system operating using a first operating configuration.

2. The method of claim 1, wherein the method further comprises:
requesting a control setting change associated with the first operating configuration used by the first surgical system, wherein the request indicates to use a second operating configuration; and
determining that the first surgical system is using a second operating configuration based on a received acknowledgement (ACK).

3. The method of claim 1, wherein the support and control information indicates a plurality of operating configurations associated with the first surgical system that the second surgical system is enabled to change.

4. The method of claim 1, wherein the support and control information indicates a degree of control associated with the second surgical system changing an operating configuration associated with the first surgical system, wherein the degree of control is one of partial control or full control.

5. The method of claim 1, wherein the method further comprises:
determining that the first surgical system switched from the first operating configuration to a second operating configuration; and
displaying updated imaging from the first surgical system and displaying an updated control interface associated with the first surgical system, wherein the updated imaging and updated control interface is associated with the first surgical system operating using the second operating configuration.

6. The method of claim 1, wherein the method further comprises:
requesting a control setting change associated with the first operating configuration used by the first surgical system, wherein the request indicates to use a second operating configuration;
receiving a negative acknowledgement (NACK) based on the requested control setting change, wherein the NACK indicates a reason for refusing the control setting change request; and
updating control settings associated with remotely operating the first surgical system based on the received NACK.

7. The method of claim 1, wherein the method further comprises:
receiving an indication that indicates the first surgical system is taking control and terminating remote control of the first surgical system via the second surgical system; and
based on receiving the indication, updating control settings associated remotely operating the first surgical system.

8. A method for operating a first surgical system, the method comprising:
obtaining support and control information associated with a second surgical system;
determining, based on the support and control information, that the second surgical system is enabled to support image porting and remote control;
sending, based on the determination that the second surgical system supports image porting and remote control, imaging and control information associated with the first surgical system to the second surgical system, wherein the imaging and control information is associated with a first operating configuration being used by the first surgical system;
receiving a control setting change request indicating to use a second operating configuration;
determining whether to change the operating configuration being used by the first surgical system based on the control setting change request; and
based on determination whether to change the operating configuration being used by the first surgical system, sending a control setting change response.

9. The method of claim 8, wherein the method further comprises:
based on a determination to change the operating configuration being used by the first surgical system, changing an operating parameter associated with the second configuration; and
sending a control setting change response to the second surgical system, wherein the control setting change response comprises an acknowledgement (ACK).

10. The method of claim 8, wherein the imaging and control information is first imaging and control information, wherein the control setting change response comprises second imaging and control information associated with the second operating configuration being used by the first surgical system.

11. The method of claim 8, wherein the method further comprises:
based on a determination to refrain from changing the operating configuration being used by the first surgical system, sending a control setting change response to the second surgical system, wherein the control setting change response comprises a negative acknowledgement (NACK) and an indication that indicates a reason for refusing the control setting change request.

12. The method of claim 8, wherein the support and control information indicates a plurality of operating configurations associated with the first surgical system that the second surgical system is enabled to change.

13. The method of claim 8, wherein the support and control information indicates a degree of control associated with the second surgical system changing an operating configuration associated with the first surgical system, wherein the degree of control is one of partial control or full control.

14. The method of claim 8, wherein the methods further comprises:
determining to terminate remote control operation of the first surgical system using the second surgical system;
based on the determination to terminate remote control operation of the first surgical system via the second surgical system, send a suspension indication that indicates the first surgical system is taking control and terminating remote control of the first surgical system via the second surgical system.

15. A remote first surgical system for operating a second surgical system, the remote surgical system comprising:
a processor configured to:
obtain support and control information associated with remotely operating a second surgical system;
determine, based on the support and control information, that the second surgical system supports image porting and remote control;
request, based on the determination that the second surgical system supports image porting and remote control, redirection of imaging and a control interface information from the second surgical system to the remote first surgical system;
receive, in response to the request, imaging and control information associated with the second surgical system; and
display, based on the imaging and control information, imaging from the second surgical system and displaying a control interface associated with the second surgical system, wherein the imaging from the second surgical system and the control interface associated with the second surgical system is associated with the second surgical system operating using a first operating configuration.

16. The remote first surgical system of claim 15, wherein the processor is further configured to:
request a control setting change associated with the first operating configuration used by the second surgical system, wherein the request indicates to use a second operating configuration; and
determine that the second surgical system is using a second operating configuration based on a received acknowledgement (ACK).

17. The remote first surgical system of claim 15, wherein the support and control information indicates a plurality of operating configurations associated with the second surgical system that the remote first surgical system is enabled to change, and wherein the support and control information indicates a degree of control associated with the remote first surgical system changing an operating configuration associated with the second surgical system, wherein the degree of control is one of partial control or full control.

18. The remote first surgical system of claim 15, wherein the processor is further configured to:
determine that the second surgical system switched from the first operating configuration to a second operating configuration; and
display updated imaging from the second surgical system and displaying an updated control interface associated with the second surgical system, wherein the updated imaging and updated control interface is associated with the second surgical system operating using the second operating configuration.

19. The remote first surgical system of claim 15, wherein the processor is further configured to:
request a control setting change associated with the first operating configuration used by the second surgical system, wherein the request indicates to use a second operating configuration;
receive a negative acknowledgement (NACK) based on the requested control setting change, wherein the NACK indicates a reason for refusing the control setting change request; and
update control settings associated with remotely operating the second surgical system based on the received NACK.

20. The remote first surgical system of claim 15, wherein the processor is further configured to:
receive an indication that indicates the second surgical system is taking control and terminating remote control of the second surgical system via the remote first surgical system; and
based on receiving the indication, update control settings associated remotely operating the second surgical system.
